(19) United States
(12) Patent Application Publication
(10) Pub. No.: US 2023/0024715 A1
(43) Pub. Date: Jan. 26, 2023

(54) METHODS AND COMPOSITIONS FOR DIAGNOSIS OF TUBERCULOSIS

(71) Applicants: President and Fellows of Harvard College, Cambridge, MA (US); The Brigham and Women's Hospital, Inc., Boston, MA (US)

(72) Inventors: Liangxia Xie, Boston, MA (US); David R. Walt, Boston, MA (US)

(21) Appl. No.: 17/786,121

(22) PCT Filed: Dec. 17, 2020
(86) PCT No.: PCT/US20/65541

§ 371 Date: Jun. 16, 2022

Related U.S. Application Data
(60) Provisional application No. 62/951,449, filed on Dec. 20, 2019.

(51) Int. Cl. G01N 33/569; G01N 33/543

(52) U.S. Cl. CPC G01N 33/5695; G01N 33/54386

(57) ABSTRACT

Methods and compositions are disclosed herein to detect Mycobacterium tuberculosis antigens, e.g., lipoarabinomannan (LAM) and/or Ag85B (Rv1886c), in a sample (e.g., a human urine sample) for diagnosis of tuberculosis. By developing ultrasensitive assays, both antigens are detected, with quantifiable differences levels in TB patients vs. non-TB controls.

### RELATED APPLICATION

The application claims priority to U.S. Provisional Application No. 62/951,449, filed on Dec. 20, 2019, the entire contents of which are incorporated herein by reference.

### FIELD OF THE INVENTION

The invention relates to methods for diagnosing a pathogen infection, e.g., an M. tuberculosis infection.

### BACKGROUND OF THE INVENTION

Tuberculosis (TB) is a major global health concern with high morbidity and mortality, despite being a curable disease. The primary cause of TB in humans is infection with Mycobacterium tuberculosis (“M. tuberculosis,” or “M. tb”). Despite the recognized importance of early, accurate diagnosis, about 3.0 million of the estimated 10 million new TB cases worldwide in 2018 were unreported or undiagnosed, leaving a massive diagnostic gap.

“Active” disease (active TB) is communicable and is distinguished from indolent, non-communicable infection or “latent TB.” Rapid and accurate diagnosis of active TB infection and disease is critical for timely initiation of therapeutic intervention in the infected individual. Early initiation of anti-TB drug therapy more often results in successful treatment of the infection, prevention of the infected person spreading the disease to others, and, ultimately, control of the disease in the larger community. Distinguishing those with latent TB from previously infected persons having circulating antibodies who have completely cleared the infection following treatment is also important. Treatment of latent TB may cure the infection, wherein unnecessary drug therapy in non-infected individuals contributes to the spread of drug-resistant strains of M. tuberculosis, particularly, multiple drug-resistant strains.

There are currently several methods of diagnosing M. tb infection. The diagnostic accuracy of these tests, however, differs and is highly affected by multiple host-related factors. The gold-standard for diagnosis of active TB is detection of M. tb by sputum culture. In resource-limited settings, sputum smear microscopy (SSM), acid-fast staining of M. tb tubercle bacillus, is widely used. Although SSM is highly specific for active TB, it has a sensitivity of less than 70%. The sensitivity of sputum M. tb nucleic acid amplification-based tests is higher than SSM, but is less specific for active TB infection. Non-sputum based tests include the tuberculin skin test (TST) and gamma interferon (IFN-7) release assays (IGRA) which are used to measure a host immune response to M. tb antigens but are not useful in distinguishing active TB from latent (non-communicable) disease. Vaccination with the Bacillus Calmette-Gudrin (BCG), common in TB-endemic countries, can, however, cause false positive TST results. Additionally, infection with human immunodeficiency virus (HIV), which often increases the severity of each disease in a patient co-infected with both pathogens, decreases diagnostic accuracy by affecting the sensitivity, specificity, or both, depending on the diagnostic test.

The lack of a sensitive test for TB that is specific for active (infectious) disease, regardless of previous BCG vaccination, latent non-communicable infection, or concurrent HIV infection, contributes substantially to difficulties in reducing the prevalence of this devastating disease worldwide. Accordingly, there remains a need in the art for a highly sensitive diagnostic test for M. tuberculosis infection.

### SUMMARY OF THE INVENTION

The present invention provides novel direct detection diagnostic assays, e.g., urine-based diagnostic assays, with superior sensitivity, to reliably quantify antigens expressed by M. tuberculosis (M. tb) in humans. These detection assays are able to classify active TB subjects from NTB (Non-TB) controls, and are insensitive to the effects of HIV status, demonstrating the potential for the generalized applicability of these biomarkers in the field. Specifically, the present invention can be applied to TB subjects with negative HIV status (TB+HIV−), whereas currently existing lateral flow assays are only recommended to be used within HIV+ populations. In addition to improve the detection sensitivity, the diagnostic assays of the present invention also offer a clear path to meet both practicality and cost requirements.

Accordingly, disclosed herein are methods to directly quantify Mycobacterium tuberculosis (M. tb) antigens in a sample, e.g., a urine sample, using an ultrasensitive detection method, e.g., a bead-based technology, e.g., a Single Molecule Array (Simoa) technology. The Simoa platform provides a 50 to 1000-fold increase in sensitivity for protein detection, compared to conventional enzyme-linked immunosorbent assay (ELISA), utilizing a bead-based immunoassay format with multiplex ability.

In one aspect, the current disclosure provides a method for detecting an antigen expressed by Mycobacterium tuberculosis in a subject sample containing the antigen, the method comprising: (a) contacting a subject sample containing the antigen with a plurality of capture objects, wherein each capture object comprises a capture probe coupled to a plurality of capture moieties that specifically bind to the antigen, wherein the subject sample and the plurality of capture objects are contacted at a ratio that allows for a single antigen to bind to a single capture object; (b) incubating the product of step (a) for a sufficient time to allow binding of the plurality of capture objects to the antigen contained in the sample, thereby creating a complex of the capture object and the antigen; (c) contacting the complex of the capture object and the antigen from step (b) with a plurality of detection probes, wherein the detection probes bind to the antigen within the complex of the capture object and the antigen from step (b); (d) labeling the product of step (c) with a detectable moiety; and (e) detecting the detectable moiety, thereby detecting the antigen in the sample.

In some embodiments, the Mycobacterium tuberculosis infection is an active infection. In other embodiments, the Mycobacterium tuberculosis infection is a latent infection.

In one embodiment, the method further comprises detecting additional antigens in a subject sample.

In one embodiment, the method further comprises contacting one or more groups of capture objects with a subject sample containing an antigen expressed by Mycobacterium tuberculosis, wherein each group of capture objects comprises a plurality of capture moieties that bind to a distinct epitope on the antigen. Each group of capture objects binds to one distinct epitope on the antigen, and each antigen is bound to one capture object.

In one embodiment, one or more groups of complexes of the capture object and the antigen are formed, wherein each single antigen is bound to a single capture object that binds to a distinct epitope on the antigen.

In one embodiment, each group of capture objects is distinguishably labeled by a fluorescent label or an enzymatic label.

In one embodiment, the method comprises contacting three groups of capture objects with the subject sample, wherein the three groups of capture objects bind to three distinct epitopes on the antigen. Each group of capture objects binds to one distinct epitope on the antigen in the antigen, and each antigen is bound to one capture object.

In one embodiment, three groups of complexes of the capture object and the antigen are formed, wherein each single antigen is bound to a single capture object that binds to a distinct epitope on the antigen in the antigen.

In one embodiment, each capture probe is coupled to about 1 to about 1,000,000 capture moieties.

In some embodiments, the plurality of capture objects and the antigens are incubated at a ratio of about 1:100,000 to about 50:1. In one embodiment, the plurality of capture objects and the antigens are incubated at a ratio of about 1:90,000, about 1:80,000, about 1:70,000, about 1:60,000, about 1:50,000, about 1:40,000, about 1:30,000, about 1:20,000, about 1:10,000, about 1:9000, about 1:8000; about 1:7000, about 1:6000, about 1:5000, about 1:4000; about 1:3000; about 1:2000, about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, or about 1:5. In one embodiment, the plurality of capture objects and the antigens are incubated at a ratio of about 1:100,000. In one embodiment, the plurality of capture objects and the antigens are incubated at a ratio of about 1:30.

In one embodiment, at least some of the capture objects bind to antigens and a statistically significant fraction of the capture objects do not bind with any antigen.

In one embodiment, at least 90% of the complexes of the capture object and the antigen have a single antigen bound to a single capture moiety on each capture probe.

In one aspect, the ratio of the plurality of capture objects and the antigens are adjusted to allow more than one antigens to bind to a single capture object. In one embodiment, the plurality of capture objects and the antigens are incubated at a ratio of about 1:100,000 to about 50:1. In one embodiment, the plurality of capture objects and the antigens are incubated at a ratio of about 1:30 to about 50:1.

In one embodiment, at least 70% of the capture objects are bound to one or more antigens.

In one embodiment, the incubating in step (b) is performed for about 1 minute to about 24 hours.

In one embodiment, the method further comprises a washing step after step (b) that removes excess subject sample.

In one embodiment, the method further comprises quantifying the antigen in the sample by determining the number of antigens bound to the capture objects.

In one embodiment, the method comprises contacting the subject sample with about 10,000 to about 2,000,000 capture objects.

In one embodiment, the antigen molecule expressed by Mycobacterium tuberculosis is one or more antigens selected from the group consisting of lipoarabinomannan (LAM), RelG, McelA, GlcB, HspX, CFP10, Ag85A, Ag85B, Ag85C, Adk, and GroES.

In one embodiment, the antigen molecule expressed by Mycobacterium tuberculosis is one or more antigens selected from the group consisting of lipoarabinomannan (LAM) and Ag85B (Rv1886c).

In one embodiment, the capture moiety on the capture probe is a full-length antibody, an antigen-binding fragment of an antibody, or an antibody mimetic.

In one embodiment, the full-length antibody is an IgG, IgA, IgD, IgE, or IgM antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the capture probe is selected from the group consisting of beads, nanotubes, and polymers.

In one embodiment, the capture probe is a paramagnetic bead.

In one embodiment, the bead has a diameter of about 1 m to about 5 km.

In one embodiment, the detection probe is an antibody, an aptamer, an antibody mimetic, a polypeptide, a nucleic acid, a molecularly-imprinted polymer, a receptor, or a small molecule.

In one embodiment, the detection probe is a full-length antibody, an antigen-binding fragment of an antibody, or an antibody mimetic.

In one embodiment, the full-length antibody is an IgG, IgA, IgD, IgE, or IgM antibody.

In one embodiment, the full-length antibody is a mouse anti-human IgG antibody, a rat anti-human IgG antibody, a goat anti-human IgG antibody, a sheep anti-human IgG antibody, a horse anti-human IgG antibody, or a rabbit anti-human IgG antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the detectable moiety is, or comprises, an enzymatic label, a fluorescent label, a radioactive label, or a metal label.

In one embodiment, the detectable moiety is, or comprises, an enzymatic label.

In one embodiment, the enzymatic label is selected from the group consisting of beta-galactosidase (β-galactosidase), horseradish peroxidase, glucose oxidase, and alkaline phosphatase.

In one embodiment, step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

In one embodiment, the non-covalent affinity binding pair is biotin-streptavidin, biotin-avidin, ligand-receptor, antigen-antibody, or antibody binding protein-antibody.

In one embodiment, the non-covalent affinity binding pair is biotin-streptavidin.

In one embodiment, the sample is a biological sample.

In one embodiment, the biological sample is a liquid biological sample.

In one embodiment, the sample is selected from the group consisting of lymph, serum, plasma, whole blood, urine, saliva, semen, synovial fluid, cerebrospinal fluid, sputum, mucous, feces, and vaginal fluid.

In one embodiment, the sample is a urine sample.

In one embodiment, the sample is subject to centrifugation prior to contacting with the capture objects. In another embodiment, prior to contacting with the capture objects, the sample is subject to heat treatment followed by centrifugation. In yet another embodiment, prior to contacting with the capture objects, the sample is subject to centrifugation followed by heat treatment.

In one embodiment, the method further comprises determining the HIV status of the subject. In one embodiment, the subject is a human subject. In one embodiment, the subject is HIV negative (HIV−). In another embodiment, the subject is HIV positive (HIV+).

In one embodiment, the detection of step (e) comprises single-molecule detection of the detectable moiety.

In one embodiment, the detection of step (e) occurs in an array of microwells, wherein the microwells are capable of holding zero or one capture objects.

In one embodiment, detectable moiety is detected in a fluid volume of about 10 attoliters (aL) to about 10 picoliters (pL) in a microwell.

In one embodiment, the array is a QUANTERIX™ single molecule array (Simoa).

In one embodiment, the microwells have a volume of about 40 femtoliters (fL).

In one embodiment, the detection of step (e) occurs in a plurality of water-in-oil droplets.

In one embodiment, essentially all of the droplets include zero or one capture object.

In one embodiment, the detectable moiety is detected by the presence of a fluorescent signal.

In another embodiment, the detection of step (e) comprises detecting one or more antigens bound to the plurality of capture objects.

In one embodiment, multiple detectable moieties on the detection probes bound to the one or more antigens are detected on a single capture object.

In one embodiment, the multiple detectable moieties are detected by the intensity of the fluorescent signal of each of the capture objects.

In one aspect, the current disclosure provides a method for determining the concentration of an antigen expressed by Mycobacterium tuberculosis in a subject sample containing the antigen, the method comprising (a) contacting a subject sample containing the antigen with a plurality of capture objects, wherein each capture object comprises a capture probe coupled to a plurality of capture moieties that specifically bind to the antigen, wherein the subject sample and the plurality of capture objects are contacted at a ratio that allows for a single antigen to bind to a single capture object; (b) incubating the product of step (a) for a sufficient time to allow binding of the plurality of capture objects to the antigen contained in the sample, thereby creating a complex of the capture object and the antigen; (c) contacting the complex of the capture object and the antigen from step (b) with a plurality of detection probes, wherein the detection probes bind to the antigen within the complex of the capture object and the antigen from step (b); (d) labeling the product of step (c) with a detectable moiety; and (e) detecting the detectable moiety; and (f) quantifying the concentration of the antigen in the subject sample by determining the number of detected moieties in step (e), thereby determining the concentration of the antigen in the sample.

In some embodiments, the Mycobacterium tuberculosis infection is an active infection. In other embodiments, the Mycobacterium tuberculosis infection is a latent infection.

In one embodiment, each capture probe is coupled to about 1 to about 1,000,000 capture moieties.

In some embodiments, the plurality of capture objects and the antigens are incubated at a ratio of about 1:100,000 to about 50:1. In one embodiment, the plurality of capture objects and the antigens are incubated at a ratio of about 1:90,000, about 1:80,000, about 1:70,000, about 1:60,000, about 1:50,000, about 1:40,000, about 1:30,000, about 1:20,000, about 1:10,000, about 1:9000, about 1:8000; about 1:7000, about 1:6000, about 1:5000, about 1:4000; about 1:3000; about 1:2000, about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, or about 1:5. In one embodiment, the plurality of capture objects and the antigens are incubated at a ratio of about 1:100,000. In one embodiment, the plurality of capture objects and the antigens are incubated at a ratio of about 1:30.

In one embodiment, at least some of the capture objects bind to antigens and a statistically significant fraction of the capture objects do not bind with any antigen.

In one embodiment, at least 90% of the complexes of the capture object and the antigen have a single antigen bound to a single capture moiety on each capture probe.

In one aspect, the ratio of the plurality of capture objects and the antigens are adjusted to allow more than one antigens to bind to a single capture object. In one embodiment, the plurality of capture objects and the antigens are incubated at a ratio of about 1:100,000 to about 50:1. In one embodiment, the plurality of capture objects and the antigens are incubated at a ratio of about 1:30 to about 50:1.

In one embodiment, at least 70% of the capture objects are bound to one or more antigens.

In one embodiment. The incubating in step (b) is performed for about 1 minute to about 24 hours.

In one embodiment, the method further comprises a washing step after step (b) that removes excess subject sample.

In one embodiment, the method comprises contacting the subject sample with about 10,000 to about 2,000,000 capture objects.

In one embodiment, the method comprises contacting one or more groups of capture objects with a subject sample containing an antigen expressed by Mycobacterium tuberculosis, wherein each group of capture objects comprises a plurality of capture moieties that bind to a distinct epitope on the antigen.

In one embodiment, each group of capture objects is distinguishably labeled by a fluorescent label or an enzymatic label.

In one embodiment, the method comprises contacting three groups of capture objects with the subject sample, wherein the three groups of capture objects bind to three distinct epitopes on the antigen.

In one embodiment, the antigen molecule expressed by Mycobacterium tuberculosis is one or more antigens selected from the group consisting of lipoarabinomannan (LAM) and Ag85B (Rv1886c).

In one embodiment, the capture moiety on the capture probe is a full-length antibody, an antigen-binding fragment of an antibody, or an antibody mimetic.

In one embodiment, the full-length antibody is an IgG, IgA, IgD, IgE, or IgM antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the capture probe is selected from the group consisting of beads, nanotubes, and polymers.

In one embodiment, the capture probe is a paramagnetic bead.

In one embodiment, the bead has a diameter of about 1 m to about 5 km.

In one embodiment, the detection probe is an antibody, an aptamer, an antibody mimetic, a polypeptide, a nucleic acid, a molecularly-imprinted polymer, a receptor, or a small molecule.

In one embodiment, the detection probe is a full-length antibody, an antigen-binding fragment of an antibody, or an antibody mimetic.

In one embodiment, the full-length antibody is an IgG, IgA, IgD, IgE, or IgM antibody.

In one embodiment, the full-length antibody is a mouse anti-human IgG antibody, a rat anti-human IgG antibody, a goat anti-human IgG antibody, a sheep anti-human IgG antibody, a horse anti-human IgG antibody, or a rabbit anti-human IgG antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the detectable moiety is, or comprises, an enzymatic label, a fluorescent label, a radioactive label, or a metal label.

In one embodiment, the detectable moiety is, or comprises, an enzymatic label.

In one embodiment, the enzymatic label is selected from the group consisting of beta-galactosidase (β-galactosidase), horseradish peroxidase, glucose oxidase, and alkaline phosphatase.

In one embodiment, step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

In one embodiment, the non-covalent affinity binding pair is biotin-streptavidin, biotin-avidin, ligand-receptor, antigen-antibody, or antibody binding protein-antibody.

In one embodiment, the non-covalent affinity binding pair is biotin-streptavidin.

In one embodiment, the sample is a biological sample.

In one embodiment, the biological sample is a liquid biological sample.

In one embodiment, the sample is selected from the group consisting of lymph, serum, plasma, whole blood, urine, saliva, semen, synovial fluid, cerebrospinal fluid, sputum, mucous, feces, and vaginal fluid.

In one embodiment, the sample is a urine sample.

In one embodiment, the sample is subject to centrifugation prior to contacting with the capture objects. In another embodiment, prior to contacting with the capture objects, the sample is subject to heat treatment followed by centrifugation. In yet another embodiment, prior to contacting with the capture objects, the sample is subject to centrifugation followed by heat treatment.

In one embodiment, the method further comprises determining the HIV status of the subject. In one embodiment, the subject is a human subject. In one embodiment, the subject is HIV negative (HIV−). In another embodiment, the subject is HIV positive (HIV+).

In one embodiment, the detection of step (e) comprises single-molecule detection of the detectable moiety.

In one embodiment, the detection of step (e) occurs in an array of microwells, wherein the microwells are capable of holding zero or one capture objects.

In one embodiment, the detectable moiety is detected in a fluid volume of about 10 attoliters (aL) to about 10 picoliters (pL) in a microwell.

In one embodiment, the array is a QUANTERIX™ single molecule array (Simoa).

In one embodiment, the microwells have a volume of about 40 femtoliters.

In one embodiment, the detection of step (e) occurs in a plurality of water-in-oil droplets.

In one embodiment, essentially all of the droplets includes zero or one capture object.

In one embodiment, the detectable moiety is detected by the presence of fluorescent signals.

In another embodiment, the detection of step (e) comprises detecting one or more antigens bound to the plurality of capture objects.

In one embodiment, multiple detectable moieties on the detection probes bound to the one or more antigens are detected on a single capture object.

In one embodiment, the multiple detectable moieties are detected by the intensity of the fluorescent signal of each of the capture objects.

In one aspect, the current disclosure provides a method for diagnosing a Mycobacterium tuberculosis infection in a subject, the method comprising (a) contacting a subject sample suspected of containing an antigen expressed by Mycobacterium tuberculosis with a plurality of capture objects, wherein each capture object comprises a capture probe coupled to a plurality of capture moieties that specifically bind to the antigen, wherein the subject sample and the plurality of capture objects are contacted at a ratio that allows for a single antigen to bind to a single capture object; (b) incubating the product of step (a) for a sufficient time to allow binding of the plurality of capture objects to the antigen contained in the sample, thereby creating a plurality of complexes of the capture object and the antigen; (c) contacting the plurality of complex of the capture object and the antigen from step (b) with a plurality of detection probes, wherein the detection probes bind to the antigen within the complex of the capture object and the antigen from step (b); (d) labeling the product of step (c) with a detectable moiety; (e) detecting the detectable moiety; (f) quantifying the concentration of the antigen in the subject sample by determining the number of detected moieties in step (e); and (g) comparing the concentration determined in step (f) with a control value determined using a sample from a healthy subject, wherein an antigen concentration greater than the control value indicates the presence of Mycobacterium tuberculosis infection in the subject and an antigen concentration less than the control value indicates the absence of Mycobacterium tuberculosis infection in the subject, thereby diagnosing the Mycobacterium tuberculosis infection in the subject.

In one embodiment, the Mycobacterium tuberculosis infection is an active infection.

In one embodiment, the Mycobacterium tuberculosis infection is a latent infection.

In another aspect, the present invention provides a method for diagnosing a Mycobacterium tuberculosis infection in a subject, the method comprising detecting at least two antigen molecules expressed by Mycobacterium tuberculosis in a subject sample. The methods include (1) contacting a subject sample containing the first antigen molecules with a plurality of first capture objects, wherein each first capture object comprises a first capture probe coupled to a plurality of first capture moieties that specifically bind to the first antigen molecules expressed by M. tb, wherein the subject sample and the plurality of the first capture objects are contacted at a ratio that allows for a single first antigen molecule to bind to a single first capture object; incubating the first capture objects and the first antigen molecules for a sufficient time to allow binding of the plurality of first capture objects to the first antigen molecules contained in the sample, thereby creating a first capture object-antigen molecule complex; contacting the first capture object-antigen molecule complex from the previous step with a plurality of first detection probes, wherein the first detection probe binds to the first antigen molecule within the first capture object-antigen molecule complex; labeling the first product with a first detectable moiety; and detecting the first detectable moiety; and (2) contacting the subject sample containing the second antigen molecules with a plurality of second capture objects, wherein each second capture object comprises a second capture probe coupled to a plurality of second capture moieties that specifically bind to the second antigen molecules expressed by M. tb, wherein the subject sample and the plurality of second capture objects are contacted at a ratio that allows for a single second antigen molecule to bind to a single second capture object; incubating the second capture objects and the second antigen molecules for a sufficient time to allow binding of the plurality of second capture objects to the second antigen molecules contained in the sample, thereby creating a second capture object-antigen molecule complex; contacting the second capture object-antigen molecule complex from the previous step with a plurality of second detection probes, wherein the second detection probe binds to the second antigen molecule within the second capture object-antigen molecule complex; labeling the second product with a second detectable moiety; and detecting the second detectable moiety.

In some embodiments, the methods further comprise quantifying the concentration of the first antigen and the second antigen in the subject sample by determining the number of the first detected moieties and the second detected moieties, and comparing the concentration of the first antigen and the second antigen with a control value determined using a sample from a healthy subject, wherein a concentration of the first antigen and the second antigen greater than the control value indicates the presence of Mycobacterium tuberculosis infection in the subject and a concentration of the first antigen and the second antigen less than the control value indicates the absence of Mycobacterium tuberculosis infection in the subject, thereby diagnosing the Mycobacterium tuberculosis infection in the subject.

In one embodiment, the first detectable moiety and the second detectable moiety are the same. In another embodiment, the first detectable moiety and the second detectable moiety are different.

In one embodiment, the antigen molecule expressed by Mycobacterium tuberculosis is one or more antigens selected from the group consisting of lipoarabinomannan (LAM) and Ag85B (Rv1886c).

In one embodiment, each capture probe is coupled to about 1 to about 1,000,000 capture moieties.

In some embodiments, the plurality of capture objects and the antigens are incubated at a ratio of about 1:100,000 to about 50:1. In one embodiment, the plurality of capture objects and the antigens are incubated at a ratio of about 1:90,000, about 1:80,000, about 1:70,000, about 1:60,000, about 1:50,000, about 1:40,000, about 1:30,000, about 1:20,000, about 1:10,000, about 1:9000, about 1:8000; about 1:7000, about 1:6000, about 1:5000, about 1:4000; about 1:3000; about 1:2000, about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, or about 1:5. In one embodiment, the plurality of capture objects and the antigens are incubated at a ratio of about 1:100,000. In one embodiment, the plurality of capture objects and the antigens are incubated at a ratio of about 1:30.

In one embodiment, at least some of the capture objects bind to antigen molecules and a statistically significant fraction of the capture objects do not bind with any antigen molecule.

In one embodiment, at least 90% of the capture object-antigen molecule complexes have a single antigen molecule bound to a single capture moiety on each capture probe.

In one aspect, the ratio of the plurality of capture objects and the antigens are adjusted to allow more than one antigens to bind to a single capture object. In one embodiment, the plurality of capture objects and the antigens are incubated at a ratio of about 1:100,000 to about 50:1. In one embodiment, the plurality of capture objects and the antigens are incubated at a ratio of about 1:30 to about 50:1.

In one embodiment, at least 70% of the capture objects are bound to one or more antigens.

In one embodiment, the incubating in step (b) is performed for about 1 minute to about 24 hours.

In one embodiment, the method further comprises a washing step after step (b) that removes excess subject sample.

In one embodiment, the method comprises contacting the subject sample with about 10,000 to about 2,000,000 capture objects.

In one embodiment, the method comprises contacting one or more groups of capture objects with a subject sample containing an antigen expressed by Mycobacterium tuberculosis, wherein each group of capture objects comprises a plurality of capture moieties that bind to a distinct epitope on the antigen.

In one embodiment, each group of capture objects is distinguishably labeled by a fluorescent label or an enzymatic label.

In one embodiment, the method comprises contacting three groups of capture objects with the subject sample, wherein the three groups of capture objects bind to three distinct epitopes on the antigen.

In one embodiment, an antigen molecule concentration greater than the control value indicates active tuberculosis (TB).

In one embodiment, an antigen molecule concentration less than the control value indicates absence of active or latent TB.

In one embodiment, the method further comprises determining the HIV status of the subject. In one embodiment, the subject is a human subject. In one embodiment, the subject is HIV negative (HIV−) or HIV positive (HIV+).

In one embodiment, the subject has been vaccinated with a Bacille Calmette-Guerin vaccine.

In one embodiment, the antigen molecule expressed by Mycobacterium tuberculosis is one or more antigens selected from the group consisting of lipoarabinomannan (LAM) and Ag85B (Rv1886c).

In one embodiment, the capture moiety on the capture probe is a full-length antibody, an antigen-binding fragment of an antibody, or an antibody mimetic.

In one embodiment, the full-length antibody is an IgG, IgA, IgD, IgE, or IgM antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the capture probe is selected from the group consisting of beads, nanotubes, and polymers.

In one embodiment, the capture probe is a paramagnetic bead.

In one embodiment, the bead has a diameter of about 1 m to about 5 km.

In one embodiment, the detection probe is an antibody, an aptamer, an antibody mimetic, a polypeptide, a nucleic acid, a molecularly-imprinted polymer, a receptor, or a small molecule.

In one embodiment, the detection probe is a full-length antibody, an antigen-binding fragment of an antibody, or an antibody mimetic.

In one embodiment, the full-length antibody is an IgG, IgA, IgD, IgE, or IgM antibody.

In one embodiment, the full-length antibody is a mouse anti-human IgG antibody, a rat anti-human IgG antibody, a goat anti-human IgG antibody, a sheep anti-human IgG antibody, a horse anti-human IgG antibody, or a rabbit anti-human IgG antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the detectable moiety is, or comprises, an enzymatic label, a fluorescent label, a radioactive label, or a metal label.

In one embodiment, the detectable moiety is, or comprises, an enzymatic label.

In one embodiment, the enzymatic label is selected from the group consisting of beta-galactosidase (β-galactosidase), horseradish peroxidase, glucose oxidase, and alkaline phosphatase.

In one embodiment, step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

In one embodiment, the non-covalent affinity binding pair is biotin-streptavidin, biotin-avidin, ligand-receptor, antigen-antibody, or antibody binding protein-antibody.

In one embodiment, the non-covalent affinity binding pair is biotin-streptavidin.

In one embodiment, the sample is a biological sample.

In one embodiment, the biological sample is a liquid biological sample.

In one embodiment, the sample is selected from the group consisting of lymph, serum, plasma, whole blood, urine, saliva, semen, synovial fluid, cerebrospinal fluid, sputum, mucous, feces, and vaginal fluid.

In one embodiment, the sample is a urine sample.

In one embodiment, the sample is subject to centrifugation prior to contacting with the capture objects. In another embodiment, prior to contacting with the capture objects, the sample is subject to heat treatment followed by centrifugation. In yet another embodiment, prior to contacting with the capture objects, the sample is subject to centrifugation followed by heat treatment.

In one embodiment, the method further comprises determining the HIV status of the subject. In one embodiment, the subject is a human subject. In one embodiment, the subject is HIV negative (HIV−). In another embodiment, the subject is HIV positive (HIV+).

In one embodiment, the detection of step (e) comprises single-molecule detection of the detectable moiety.

In one embodiment, the detection of step (e) occurs in an array of microwells, wherein the microwells are capable of holding zero or one capture objects.

In one embodiment, the detectable moiety is detected in a fluid volume of about 10 attoliters (aL) to about 10 picoliters (pL) in a microwell.

In one embodiment, the array is a QUANTERIX™ single molecule array (Simoa).

In one embodiment, the microwells have a volume of about 40 femtoliters.

In one embodiment, the detection of step (e) occurs in a plurality of water-in-oil droplets.

In one embodiment, essentially all of the droplets include zero or one capture object.

In one embodiment, the detectable moiety is detected by the presence of fluorescent signals.

In another embodiment, the detection of step (e) comprises detecting one or more antigens bound to the plurality of capture objects.

In one embodiment, multiple detectable moieties on the detection probes bound to the one or more antigens are detected on a single capture object.

In one embodiment, the multiple detectable moieties are detected by the intensity of the fluorescent signal of each of the capture objects.

In one aspect, the current disclosure provides a method for treating a Mycobacterium tuberculosis infection in a subject, the method comprising (a) contacting a subject sample containing an antigen expressed by Mycobacterium tuberculosis with a plurality of capture objects, wherein each capture object comprises a capture probe coupled to a plurality of capture moieties that specifically bind to the antigen, wherein the subject sample and the plurality of capture objects are contacted at a ratio that allows for a single antigen to bind to a single capture object; (b) incubating the product of step (a) for a sufficient time to allow binding of the plurality of capture objects to the antigen contained in the sample, thereby creating a plurality of complexes of the capture object and the antigen; (c) contacting the plurality of complex of the capture object and the antigen from step (b) with a plurality of detection probes, wherein the detection probes bind to the antigen within the complex of the capture object and the antigen from step (b); (d) labeling the product of step (c) with a detectable moiety; (e) detecting the detectable moiety; (f) quantifying the concentration of the antigen in the subject sample by determining the number of detected moieties in step (e); (g) comparing the concentration determined in step (f) with a control value determined using a sample from a healthy subject, wherein an antigen concentration greater than the control value indicates the presence of Mycobacterium tuberculosis infection in the subject and an antigen concentration less than the control value indicates the absence of Mycobacterium tuberculosis infection in the subject; and (h) administering an anti-Mycobacterium tuberculosis treatment to the subject diagnosed with a Mycobacterium tuberculosis infection, thereby treating the Mycobacterium tuberculosis infection in the subject.

In one aspect, the current disclosure provides a method for monitoring the treatment of a Mycobacterium tuberculosis infection in a subject, the method comprising (a) contacting a subject sample suspected of containing an antigen expressed by Mycobacterium tuberculosis with a plurality of capture objects, wherein each capture object comprises a capture probe coupled to a plurality of capture moieties that specifically bind to the antigen, wherein the subject sample and the plurality of capture objects are contacted at a ratio that allows for a single antigen to bind to a single capture object; (b) incubating the product of step (a) for a sufficient time to allow binding of the plurality of capture objects to the antigen contained in the sample, thereby creating a plurality of complexes of the capture object and the antigen; (c) contacting the plurality of complex of the capture object and the antigen from step (b) with a plurality of detection probes, wherein the detection probes bind to the antigen within the complex of the capture object and the antigen from step (b); (d) labeling the product of step (c) with a detectable moiety; (e) detecting the detectable moiety; (f) quantifying the concentration of the antigen in the subject sample by determining the number of detected moieties in step (e); (g) comparing the concentration determined in step (f) with a control value determined using a sample from a healthy subject, wherein an antigen concentration greater than the control value indicates the presence of Mycobacterium tuberculosis infection in the subject and an antigen concentration less than the control value indicates the absence of Mycobacterium tuberculosis infection in the subject; (h) administering an anti-Mycobacterium tuberculosis treatment to the subject diagnosed with a Mycobacterium tuberculosis infection; and (i) determining the concentration of the antigen after step (h), and comparing the concentration determined in step (h) with the antigen concentration prior to the anti-Mycobacterium tuberculosis treatment, thereby monitoring the treatment of Mycobacterium tuberculosis infection in the subject.

In one embodiment, the Mycobacterium tuberculosis infection is an active infection.

In one embodiment, the Mycobacterium tuberculosis infection is a latent infection.

In one embodiment, each capture probe is coupled to about 1 to about 1,000,000 capture moieties.

In some embodiments, the plurality of capture objects and the antigens are incubated at a ratio of about 1:100,000 to about 50:1. In one embodiment, the plurality of capture objects and the antigens are incubated at a ratio of about 1:90,000, about 1:80,000, about 1:70,000, about 1:60,000, about 1:50,000, about 1:40,000, about 1:30,000, about 1:20,000, about 1:10,000, about 1:9000, about 1:8000; about 1:7000, about 1:6000, about 1:5000, about 1:4000; about 1:3000; about 1:2000, about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, or about 1:5. In one embodiment, the plurality of capture objects and the antigens are incubated at a ratio of about 1:100,000. In one embodiment, the plurality of capture objects and the antigens are incubated at a ratio of about 1:30.

In one embodiment, at least some of the capture objects bind to antigen molecules and a statistically significant fraction of the capture objects do not bind with any antigen molecule.

In one embodiment, at least 90% of the capture object-antigen molecule complexes have a single antigen molecule bound to a single capture moiety on each capture probe.

In one aspect, the ratio of the plurality of capture objects and the antigens are adjusted to allow more than one antigens to bind to a single capture object. In one embodiment, the plurality of capture objects and the antigens are incubated at a ratio of about 1:100,000 to about 50:1. In one embodiment, the plurality of capture objects and the antigens are incubated at a ratio of about 1:30 to about 50:1.

In one embodiment, at least 70% of the capture objects are bound to one or more antigens.

In one embodiment, the incubating in step (b) is performed for about 1 minute to about 24 hours.

In one embodiment, the method further comprises a washing step after step (b) that removes excess subject sample.

In one embodiment, the method comprises contacting the subject sample with about 10,000 to about 2,000,000 capture objects.

In one embodiment, the method comprises contacting one or more groups of capture objects with a subject sample containing an antigen molecule expressed by Mycobacterium tuberculosis, wherein each group of capture objects comprises a plurality of capture moieties that bind to a distinct epitope on the antigen molecule.

In one embodiment, each group of capture objects is distinguishably labeled by a fluorescent label or an enzymatic label.

In one embodiment, the method comprises contacting three groups of capture objects with the subject sample, wherein the three groups of capture objects bind to three distinct epitopes on the antigen molecule.

In one embodiment, the antigen molecule concentration is greater than the control value.

In one embodiment, the method further comprises determining the HIV status of the subject. In one embodiment, the subject is a human subject. In one embodiment, the subject is HIV negative (HIV−) or HIV positive (HIV+).

In one embodiment, the subject has been vaccinated with a Bacille Calmette-Guerin vaccine.

In one embodiment, the subject has a history of previous infection with Mycobacterium tuberculosis.

In one embodiment, the antigen molecule expressed by Mycobacterium tuberculosis is one or more antigens selected from the group consisting of lipoarabinomannan (LAM) and Ag85b (Rv1886c).

In one embodiment, the capture moiety on the capture probe is a full-length antibody, an antigen-binding fragment of an antibody, or an antibody mimetic.

In one embodiment, the full-length antibody is an IgG, IgA, IgD, IgE, or IgM antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the capture probe is selected from the group consisting of beads, nanotubes, and polymers.

In one embodiment, the capture probe is a paramagnetic bead.

In one embodiment, the bead has a diameter of about 1 m to about 5 m.

In one embodiment, the detection probe is an antibody, an aptamer, an antibody mimetic, a polypeptide, a nucleic acid, a molecularly-imprinted polymer, a receptor, or a small molecule.

In one embodiment, the detection probe is a full-length antibody, an antigen-binding fragment of an antibody, or an antibody mimetic.

In one embodiment, the full-length antibody is an IgG, IgA, IgD, IgE, or IgM antibody.

In one embodiment, the full-length antibody is a mouse anti-human IgG antibody, a rat anti-human IgG antibody, a goat anti-human IgG antibody, a sheep anti-human IgG antibody, a horse anti-human IgG antibody, or a rabbit anti-human IgG antibody.

In one embodiment, the antigen-binding fragment is an scFv, an Fv, a dAb, a Fab, an Fab′, an Fab′2, an F(ab′)2, an Fd, an Fv, or an Feb.

In one embodiment, the antibody mimetic is an affibody, an affilin, an affimer, an affitin, an alphabody, an anticalin, an avimer, a DARPin, a fynomer, a Kunitz domain peptide, a monobody, or a nanoCLAMP.

In one embodiment, the detectable moiety is, or comprises, an enzymatic label, a fluorescent label, a radioactive label, or a metal label.

In one embodiment, the detectable moiety is, or comprises, an enzymatic label.

In one embodiment, the enzymatic label is selected from the group consisting of beta-galactosidase (β-galactosidase), horseradish peroxidase, glucose oxidase, and alkaline phosphatase.

In one embodiment, step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

In one embodiment, the non-covalent affinity binding pair is biotin-streptavidin, biotin-avidin, ligand-receptor, antigen-antibody, or antibody binding protein-antibody.

In one embodiment, the non-covalent affinity binding pair is biotin-streptavidin.

In one embodiment, the sample is a biological sample.

In one embodiment, the biological sample is a liquid biological sample.

In one embodiment, the sample is selected from the group consisting of lymph, serum, plasma, whole blood, urine, saliva, semen, synovial fluid, cerebrospinal fluid, sputum, mucous, feces, and vaginal fluid.

In one embodiment, the sample is a urine sample.

In one embodiment, the sample is subject to centrifugation prior to contacting with the capture objects. In another embodiment, prior to contacting with the capture objects, the sample is subject to heat treatment followed by centrifugation. In yet another embodiment, prior to contacting with the capture objects, the sample is subject to centrifugation followed by heat treatment.

In one embodiment, the method further comprises determining the HIV status of the subject. In one embodiment, the subject is a human subject. In one embodiment, the subject is HIV negative (HIV−). In another embodiment, the subject is HIV positive (HIV+).

In one embodiment, the detection of step (e) comprises single-molecule detection of the detectable moiety.

In one embodiment, the detection of step (e) occurs in an array of microwells, wherein the microwells are capable of holding zero or one capture objects.

In one embodiment, the detectable moiety is detected in a fluid volume of about 10 attoliters (aL) to about 10 picoliters (pL) in a microwell.

In one embodiment, the array is a QUANTERIX™ single molecule array (Simoa).

In one embodiment, the microwells have a volume of about 40 femtoliters (fL).

In one embodiment, the detection of step (e) occurs in a plurality of water-in-oil droplets.

In one embodiment, essentially all of the droplets includes zero or one capture object.

In one embodiment, the detectable moiety is detected by the presence of fluorescent signals.

In another embodiment, the detection of step (e) comprises detecting one or more antigens bound to the plurality of capture objects.

In one embodiment, multiple detectable moieties on the detection probes bound to the one or more antigens are detected on a single capture object.

In one embodiment, the multiple detectable moieties are detected by the intensity of the fluorescent signal of each of the capture objects.

In one embodiment, the anti-Mycobacteria tuberculosis treatment is administered orally.

Additional features and advantages of the invention will be apparent from the following detailed descriptions, the drawings, and the claims.

### BRIEF DESCRIPTION OF THE DRAWINGS

FIG. 1A is a schematic illustration of representative elements of the Simoa digital single molecule assays in FIG. 1B.

FIG. 1B is a schematic illustration of Simoa digital single molecule assays for detection of a Mycobacterium tuberculosis (M. tb) antigen. Generally, methods comprise steps, including a first incubation (for example, fifteen (15) minutes) of a capture object (e.g., paramagnetic beads coated with anti-M. tb antigen antibodies) with a subject sample, followed by a second incubation (for example, five (5) minutes) with detection probes, such as a biotinylated anti-M. tb antigen antibody, and a third incubation (for example, five (5) minutes) with a detectable moiety, such a streptavidin-β-glactosidase (SBG). Beads are resuspended in substrate and loaded into microwells for imaging.

FIG. 2 is a graph of Simoa calibration curve for detection of an M. tb antigen, Ag85B, in a urine sample, plotted on a log-log scale. Ag85B standard was spiked into diluted urine mimicking buffer (75%, v/v), then serially diluted.

FIG. 3A depicts the pairwise combinations of capture beads (rows) and detection antibodies (columns) to detect 5,000 pg/ml of purified LAM from M. tb. The heat map presents the log signal-to-background (log S/B) ratio. FIG. 3B depicts the log signal ratios of M. tb over M. leprae LAM and FIG. 3C depicts the log signal ratios of M. tb over M. smegmatis LAM at the same concentration of 5,000 pg/mL. Yellow squares represent saturated signal readings of M. tb LAM.

FIGS. 4A-C depict the pairwise combinations of capture beads (rows) and detection antibodies (columns) to detect 50-fold diluted TB+HIV+ urine samples. The heat maps present the log signal-to-background (log S/B) ratios. Each heat map shows ratios for one individual sample. Crossed cells: antibody combinations not tested.

FIG. 5 depicts the effect of pre-treatment on detected LAM concentrations. Black bars: heat treatment prior to centrifuging; grey bars: centrifuging prior to heat treatment; patterned bars: centrifuge-only. Inset: coefficient of variation (CV) within three treatment procedures. Heat treatment was performed at 85° C. for 10 min. Centrifuging was performed at 10,000 g for 5 min. No significant difference is seen between the various treatments.

FIG. 6 depicts the calibration curve for LAM assay developed for detection in urine matrix. LAM standard was spiked into Quanterix Sample Diluent Buffer, then serially diluted.

FIG. 7A depicts the LAM concentrations in urine. FIG. 7B depicts Ag85B concentration in urine. Dashed line: sample limit of detection (LOD). Concentration values are only meaningful for points above the LOD. Each dot represents one subject. Open circles: HIV− subjects; full circles: HIV+ subjects. S+C+: smear+ and culture+TB+ subjects; NonTB_NonLTBI: TB− subjects without latent TB infections.

FIG. 8A depicts the heat map showing the measured Ag85B and LAM concentrations for all tested urine samples (columns). The samples are grouped by the donors' TB and HIV status, and sorted according to LAM concentrations (high to low). FIG. 8B depicts the breakdown of TB+ subjects (left) and TB− subjects (right) with different LAM/Ag85B detectabilities.

FIG. 9A depicts the study design flowchart. FIG. 9B depicts the sample cohort assignment.

FIGS. 10A and 10B depict calibration curves of all investigated markers. AEB (average enzyme per bead) is the signal unit for Simoa measures.

FIGS. 11A and 11B depict the distribution of LAM and Ag85B concentrations in overall patient cohorts and in patient cohorts stratified by HIV status. FIG. 11A depicts the model building cohort and FIG. 11B depicts the validation cohort. All patients were included and shown as individual dots in the box plots. Dotted lines represent the analytical limit of detection (LOD) of Simoa assays. The line within each box is the median value of the distribution. The box shows the interquartile range (IQR) with the values in the 25th to 75th percentile, and the “whiskers” below and above the box show the full concentration range outside the upper and lower quartiles. The tables show the total number of samples (N), number of samples that were undetectable [N(ND)] in each disease category, and the means with 95% CI and the SD of the marker concentrations in urine. The mean concentrations were calculated after assigning a value at LOD to ND samples. *95% CI lower limit below LOD values were replaced with values at LOD.

FIGS. 12A and 12B depict model training performance and threshold selection. FIG. 12A depicts ROC curve yielded from the training cohort for model building. Open circle shows the Youden threshold, and the cross shows the chosen threshold. Smaller box indicates WHO TPP optimum criteria for TB triage test (95% sensitivity, 80% specificity), and larger box indicates minimum criteria (90% sensitivity, 70% specificity). Shaded areas represent the 95% CIs of the ROC curve sensitivities, plotted at 1% specificity intervals. FIG. 12B depicts performance metrics at designated thresholds. Data are represented in percentage (%) (95% CI).

FIGS. 13A and 13B depict ROC curves of the LAM, Ag85B and HIV-status three-marker model performance in blinded validation cohort. Smaller box indicates WHO TPP optimum criteria for a TB triage test (95% sensitivity, 80% specificity), and larger box indicates the minimum criteria (90% sensitivity, 70% specificity). Shaded areas represent the 95% CIs of the ROC curve sensitivities, plotted at 1% specificity intervals. FIG. 13A depicts the overall performance in the entire validation cohort. Black-cross shows the cutoff threshold. FIG. 13B depicts performance in the HIV-positive versus HIV-negative TB patients. p-value is calculated by pairwise comparison of ROC curves in the HIV-positive and HIV-negative TB subset using DeLong tests.

FIGS. 14A and 14B depict ROC curves of model with reduced markers in validation cohort. Shaded areas represent the 95% CIs of the ROC curve sensitivities, plotted at 1% specificity intervals. FIG. 14A depicts model performance with and without HIV-status as a classifier. FIG. 14B depicts model performance without LAM or Ag85B.

FIGS. 15A and 15B depict the model performance including the smear-negative TB patients. FIG. 15A depicts the distribution of LAM and Ag85B concentrations for all patients in the study (N=418). All patients were included and shown as individual dots in the box plots. Smear-negative patients are shown as crosses. Dotted lines represent the analytical limit of detection (LOD) of Simoa assays. The line within each box is the median value of the distribution. The box shows the interquartile range (IQR) with the values in the 25th to 75th percentile, and the “whiskers” below and above the box show the full concentration range outside the upper and lower quartiles. FIG. 15B depicts the ROC curves of the three-marker model in the validation cohort without smear-negative TB patients (black line), and the validation cohort with a subset of smear-negative TB patients (dotted line). Smaller box indicates WHO TPP optimal criteria for TB triage test (95% sensitivity, 80% specificity), and larger box indicates minimum criteria (90% sensitivity, 70% specificity). Shaded areas represent the 95% CIs of the ROC curve sensitivities, plotted at 1% specificity intervals.

FIG. 16 depicts correlation of LAM and Ag85B concentrations in urine. Dotted lines represent the analytical limit of detection (LOD) of Simoa assays. Circles: TB patients, triangles: NonTB individuals, full circles/triangles: HIV-positive patients, open circles/triangles: HIV-negative individuals. Samples below LOD were assigned a value at LOD and included in the correlation analysis. Spearman correlation coefficients was calculated as 0.68 (p<0.001).

FIG. 17 depicts correlation of HIV CD4 counts with LAM and Ag85B concentrations in urine. Only HIV-positive patients with available CD4 count were included (N=190). Dotted lines represent the analytical limit of detection (LOD) of Simoa assays. Black dots TB patients, shaded dots: NonTB individuals. Samples below LOD were assigned a value at LOD and included in the correlation analysis. Spearman correlation coefficients were −0.41 (p<0.001) for CD4-LAM correlation, and −0.48 (p<0.001) for CD4-Ag85B correlation.

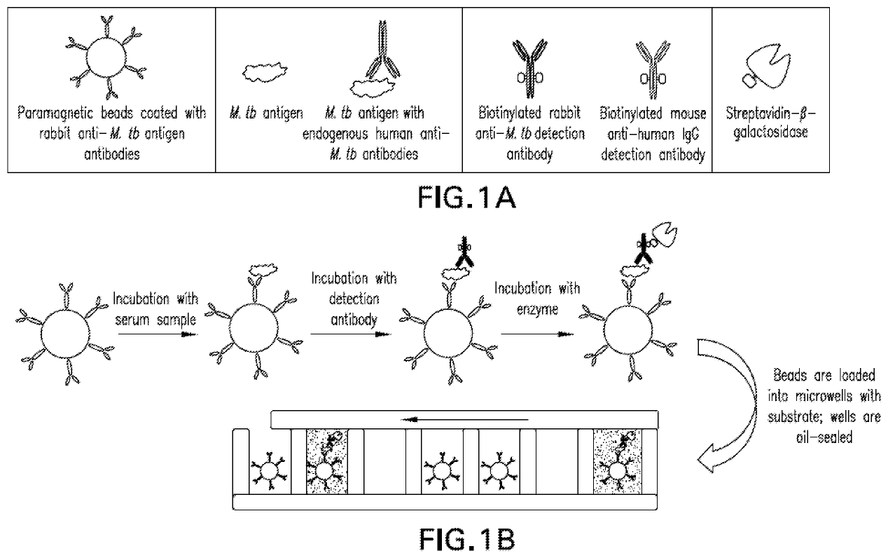

### DETAILED DESCRIPTION OF THE INVENTION

The present invention provides a platform for detecting antigen molecules expressed by Mycobacterium tuberculosis. Mycobacterium tuberculosis (M. tb) antigen molecules are promising target molecules for TB detection. By detecting and quantifying these target molecules in a sample, e.g., a urine sample, using an ultrasensitive detection method, e.g., a bead-based technology, the disclosed methods are able to diagnose a Mycobacterium tuberculosis infection in a subject, and to distinguish between TB-positive (active TB and latent TB) and non-TB subjects. Furthermore, the present invention provides methods of treatment for subjects diagnosed with active TB or latent TB, as well as methods for monitoring the treatment of these patients. The specificity and sensitivity of the disclosed methods are greatly improved by the use of ultrasensitive detection methods, e.g. a bead-based technology, as well as the detection of multiple target molecules in a sample, e.g., a combination of M. tb antigens. Importantly, the specificity and sensitivity of the disclosed methods in distinguishing latent TB from active TB from non-infection with M. tuberculosis are not substantially affected in subjects with HIV infection.

In addition, the present invention provides methods for detecting multiple and distinct epitopes of an antigen expressed by Mycobacterium tuberculosis. The ability to detect multiple epitopes on the same antigen molecule allows for more accurate and specific detection and quantification of Mycobacterium tuberculosis antigens.

### I. Definitions

In order that the present invention may be more readily understood, certain terms are first defined. It should also be noted that whenever a value or range of values of a parameter are recited, it is intended that values and ranges intermediate to the recited values are also part of this invention.

In the following description, for purposes of explanation, specific numbers, materials, and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” mean that a particular feature, structure, or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” refers to one element or more than one element.

The term “comprising” or “comprises” is used herein in reference to compositions, methods, and respective component(s) thereof, that are essential to the disclosure, yet open to the inclusion of unspecified elements, whether essential or not.

The term “portion” includes any fraction of a population (e.g., a plurality of capture objects), or region of a protein, such as a fragment of an protein (e.g., about 5%, 10%, 15%, 20%, 30%, 40%, 50%, 60%, 70%, 80%, 90%, 95%, or 99%, fewer amino acids of a full-length antibody.)

The term “antigen (Ag)” or “antigen molecule” as used herein, refers to a molecule that can induce an immune response, such as an antibody response by the host's immune system. An antigen is specifically recognized and bound by antibodies (Ab). In some embodiments, the antigen is an antigen expressed by Mycobacterium tuberculosis (M. tb), e.g., lipoarabinomannan (LAM) and Ag85B (Rv1886c).

The term “pathogen” as used herein, refers to an infectious agent, such as a bacterium, a virus, a protozoan, a prion, a viroid, or a fungus that causes disease(s). In some embodiments, a pathogen is a bacterium, e.g., an M. tb. In some embodiments, a pathogen is a virus, e.g., HIV or influenza virus. In some embodiments, a pathogen is a component or a fragment of a pathogenic agent.

The term “epitope”, as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In some embodiments, an anti-M. tb antibody recognizes and binds to an epitope on an M. tb antigen. In some embodiments, different detection probes, such as different anti-M. tb antibodies, recognize and bind to different epitopes on an M. tb antigen.

In the methods described herein, a capture object can bind to an antigen molecule, e.g., an M. tb antigen, and form a “capture object-antigen molecule complex.”

The term “capture object,” as used herein, refers to an object comprising a capture probe to which a capture moiety may be conjugated, captured, attached, bound, or affixed. Detection probes or detectable moieties may bind or otherwise associate with a capture object in single molecule array assays as described herein. Suitable capture probes include, but are not limited to, beads (e.g., paramagnetic beads), nanotubes, polymers, particulate suspensions, plates, disks, dipsticks, or the like. In some embodiments, a reaction vessel used in the methods of the invention (e.g., a microwell) is capable of holding zero or one capture object.

The term “bead” means a small discrete particle that may be used as a capture probe within the capture object. Suitable beads include, but are not limited to, paramagnetic beads, plastic beads, ceramic beads, glass beads, polystyrene beads, methylstryene beads, acrylic polymer beads, carbon graphited beads, titanium dioxide beads, latex or cross-linked dextrans such as Sepharose® beads, cellulose beads, nylon beads, cross-linked micelles, and Teflon® beads. In some embodiments, spherical beads are used, but it is to be understood that non-spherical or irregularly-shaped beads may be used.

The term “capture moiety,” as used herein, means a moiety that is capable of specifically binding to an antigen molecule expressed by M. tb.

A capture moiety may be conjugated, captured, attached, bound, or affixed to a capture probe in a capture object. For example, in some embodiments, a capture moiety is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′2, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), an antibody IgG binding protein (e.g., protein A, protein G, protein L, and recombinant protein A/G), a polypeptide, a nucleic acid, or a small molecule.

In some embodiments, a capture moiety binds to an antigen expressed by M. tb. The capture moiety is coupled to a capture probe, forming a capture object. In some embodiments, a plurality of capture moieties are coupled to a capture probe to form a capture object. In some embodiments, about 1 to about 10,000,000 capture moieties are coupled to a capture probe to form a capture object. For example, in some embodiments, about 1 to about 1,000,000 capture moieties are coupled to a capture probe to form a capture object. In some embodiments, about 500,000 to about 1,000,000 capture moieties are coupled to a capture probe to form a capture object. In some embodiments, about 10,000 to about 500,000 capture moieties are coupled to a capture probe to form a capture object.

The term “detection probe,” as used herein, refers to any molecule, particle, or the like that is capable of specifically binding to a target molecule, e.g., an antigen molecule expressed by M. tb. For example, in some embodiments, a detection probe is an antibody (e.g., a full-length antibody, such as an IgG, IgA, IgD, IgE, or IgM antibody) or an antigen-binding antibody fragment (e.g., an scFv, an Fv, a dAb, a Fab, an Fab′, an Fab′2, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly imprinted polymer, a receptor, a polypeptide, a nucleic acid, or a small molecule.

The term “detectable moiety,” as used herein, refers to a moiety that can produce a detectable signal. For example, in some embodiments, a detectable moiety is, or comprises, an enzymatic label, such as beta-galactosidase, horseradish peroxidase, glucose oxidase, and alkaline phosphatase), a fluorescent label, a radioactive label, or a metal label. In some embodiments, the detectable moiety is beta-galactosidase.

As used herein, a molecule or moiety “specifically binds” to another molecule or moiety with specificity sufficient to differentiate from other components or contaminants of the test sample. In some embodiments, a molecule or moiety specifically binds to another molecule or moiety with an equilibrium dissociation constant (K_{D}) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹ M, 10⁻¹⁴ M, 10⁻¹⁵ M, or lower. The term “non-covalent affinity binding pair” refers to a pair of moieties that bind and form a non-covalent complex. Exemplary non-covalent affinity binding pairs include, without limitation, biotin-biotin binding protein (e.g., biotin-streptavidin and biotin-avidin), ligand-receptor, antigen-antibody or antigen binding fragment, hapten-anti-hapten, and immunoglobulin (Ig) binding protein-Ig. The members of a non-covalent affinity binding pair may have any suitable binding affinity. For example, the members of an affinity binding pair may bind with an equilibrium dissociation constant (K_{D} or Kd) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹ M, 10⁻¹⁴ M, 10⁻¹ M, or lower.

As used herein, “subject” refers to any animal. In some embodiments, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, dogs, hamsters, guinea pigs, rats, mice, and birds.) As used herein, “biological sample” refers to any biological sample obtained from or derived from a subject, including body fluids, body tissue, cells, or other sources. In some embodiments, the biological sample is a liquid sample. The term “liquid sample,” as used herein, refers to a sample that is substantially in liquid form. In some embodiments, a liquid sample is a body fluid. Body fluids include, e.g., lymph, serum (including fresh or frozen), peripheral blood mononuclear cells, whole blood (including fresh or frozen), plasma (including fresh or frozen), urine (including fresh or frozen), saliva, semen, synovial fluid, cerebrospinal fluid, sputum, mucous, feces, and vaginal fluid. In some embodiments, the biological sample is a whole blood sample. In some embodiments, the biological sample is a serum sample. In some embodiments, the biological sample is a urine sample.

As used herein, a “digital enzyme-linked immunosorbent assay” and “single-molecule array assay” refer to an ultrasensitive immunoassay. These assays, as used herein, can be performed by any ultrasensitive detection method. An example of a digital enzyme-linked immunosorbent assay is the Simoa digital immunoassay. Simoa stands for “Single Molecule Array” and is a registered trademark owned by the Quanterix corporation of Billerica, Mass., U.S.A. The Simoa platform provides a 50 to 1000-fold increase in sensitivity for protein detection, compared to ELISA, utilizing a bead-based immunoassay format with multiplex ability that is amenable to implementation in future point of care (POC) devices. Unlike conventional ELISA methods, Simoa has the ability to trap single bead-bound molecules in femtoliter-sized wells, allowing for a “digital” readout of each individual bead to determine whether the bead is bound to the target molecule. The digital nature (e.g., “yes-no” or “true-false”) of the result for each well allows an average increase in sensitivity of three orders of magnitude (1,000 times) over conventional ELISA with coefficients of variation (CV) less than about ten percent.

“Tuberculosis” (“TB”) is a multisystemic disease with myriad presentations and manifestations, and is the most common cause of infectious disease-related mortality worldwide. Mycobacterium tuberculosis, a tubercle bacillus, is the causative agent of TB. The terms “Mycobacterium tuberculosis”, “M. tuberculosis”, and “M. tb” are used interchangeably herein to refer to this causative agent of TB. The lungs are the most common site for the development of TB (pulmonary TB), and about 85% of patients with TB present with pulmonary complaints. Nonetheless, “extrapulmonary TB”, e.g., “disseminated TB”, can occur as part of a primary or late, generalized infection. Extrapulmonary TB can affect bones and joints, bronchus, eye, intestines, larynx, peritoneum, meninges, pericardium, lymph node, organs of the male or female urinary and reproductive systems, skin, stomach, and/or urinary systems.

When a person is infected with M. tuberculosis, the infection can take one of a variety of paths, most of which do not lead to actual TB. The infection may be cleared by the host immune system or suppressed into an inactive form called “latent tuberculosis infection”, with resistant hosts controlling mycobacterial growth at distant foci before the development of active disease.

A subject has “latent tuberculosis (“LTB”) (also referred to as “latent tuberculosis infection” (“LTBI”)) when the subject is infected with Mycobacterium tuberculosis but does not have active tuberculosis disease. Subjects having latent tuberculosis are not infectious. The main risk is that approximately 10% of these patients (5% in the first two years after infection and 0.1% per year thereafter but higher risk if immunosuppressed) will go on to develop “active tuberculosis” (“active TB”) and spread the disease at a later stage of their life if, for example, there is onset of a disease affecting the immune system (such as AIDS) or a disease whose treatment affects the immune system (e.g., chemotherapy in cancer or systemic steroids in asthma or Enbrel, Humira or Orencia in rheumatoid arthritis); malnutrition (which may be the result of illness or injury affecting the digestive system, or of a prolonged period of not eating, or disturbance in food availability such as famine, residence in refugee camp or concentration camp, or civil war; and/or degradation of the immune system due to aging.

The symptoms of a subject having TB are similar to the symptoms of a subject having an “other respiratory disease” or “ORD”, such a pneumonia, and include, for example, cough (e.g., coughing that lasts three or more weeks, coughing up blood or sputum, chest pain, or pain with breathing or coughing), unintentional weight loss, fatigue, fever, night sweats, chills, and/or loss of appetite.

A subject having latent TB usually has a skin test or blood test result indicating TB infection; has a normal chest x-ray and a negative sputum test; has TB bacteria in his/her body that are alive, but inactive; does not feel sick (e.g. does not have a cough and/or fever); and cannot spread TB bacteria to others. A subject having active TB usually has a positive skin test or tuberculosis blood test, may have an abnormal chest x-ray, or positive sputum smear or culture; has overt indications of illness (e.g., cough and/or fever), and can spread the disease to others.

The term “HIV-positive” or “HIV+”, as used herein, refers to any patient/subject who has a positive result for human immunodeficiency virus (HIV) testing conducted at the time of TB diagnosis or other documented evidence of enrolment in HIV care, such as enrollment in the pre-antiretroviral therapy (pre-ART) register or in the antiretroviral therapy (ART) register once ART has been started.

The term “HIV-negative” or “HIV−” as used herein, refers to any patient/subject who has a negative result from HIV testing conducted at the time of TB diagnosis. Any HIV-negative patient/subject subsequently found to be HIV-positive is re-classified as an “HIV-positive subject.” In some embodiments, the HIV status (e.g., HIV-positive or HIV-negative) is used in combination with the levels of the target molecules (e.g., the M. tb antigen molecules, e.g., Ag85B and LAM) detected in the subject sample in order to determine whether a subject is infected with Mycobacterium tuberculosis.

The term “HIV status unknown” as used herein, refers to any patient/subject who has no result of HIV testing and no other documented evidence of enrollment in HIV care.

The term “CD4 cell”, also known as CD4+ T cell, refers to white blood cells that fight infection. CD4 cell count is an indicator of immune function in patients living with HIV and one of the key determinants for the need of opportunistic infection prophylaxis. CD4 cell counts are obtained from bloodwork as part of laboratory monitoring for HIV infection. CD4 cell counts show the robustness of the immune system. A healthy immune system normally has a CD4 count ranging from 500 to 1,600 cells per cubic millimeter of blood (cells/mm³). When a CD4 cell count is lower than 200 cell/mm³, patients become susceptible to infections, e.g., HIV/AIDS.

### II. Methods of the Invention

The present invention provides methods for detecting and quantifying an antigen expressed by Mycobacterium tuberculosis (M. tb) in a subject sample, e.g., a urine sample. The invention also provides methods for diagnosing a Mycobacterium tuberculosis infection in a subject and treating the subject diagnosed with the Mycobacterium tuberculosis infection.

The methods of the invention include detecting a single antigen molecule expressed by Mycobacterium tuberculosis in a subject sample. The methods include contacting a subject sample containing the antigen molecule with a plurality of capture objects, wherein each capture object comprises a capture probe coupled to a plurality of capture moieties that specifically bind to an antigen molecule expressed by M. tb, wherein the subject sample and the plurality of capture objects are contacted at a ratio that allows for a single antigen molecule to bind to a single capture object; incubating the capture objects and the M. tb antigen molecule for a sufficient time to allow binding of the plurality of capture objects to the antigen molecule contained in the sample, thereby creating a capture object-antigen molecule complex; contacting the complex from the previous step with a plurality of detection probes, wherein the detection probes bind to the antigen molecule within the capture object-antigen molecule complex; labeling the product with a detectable moiety; and detecting the detectable moiety

In some embodiments, the methods further comprise quantifying the concentration of the antigen in the subject sample by determining the number of detected moieties and comparing the concentration of the antigen with a control value determined using a sample from a healthy subject, wherein an antigen concentration greater than the control value indicates the presence of Mycobacterium tuberculosis infection in the subject and an antigen concentration less than the control value indicates the absence of Mycobacterium tuberculosis infection in the subject, thereby diagnosing the Mycobacterium tuberculosis infection in the subject.

The methods of the invention also include detecting one or more, e.g, two, three, four, five, six, seven, eight, nine, ten or eleven, antigen molecules expressed by Mycobacterium tuberculosis in a subject sample. In some embodiments, the methods of the invention comprises detecting at least two antigen molecules expressed by Mycobacterium tuberculosis in a subject sample. The methods include (1) contacting a subject sample containing the first antigen molecules with a plurality of first capture objects, wherein each first capture object comprises a first capture probe coupled to a plurality of first capture moieties that specifically bind to the first antigen molecules expressed by M. tb, wherein the subject sample and the plurality of the first capture objects are contacted at a ratio that allows for a single first antigen molecule to bind to a single first capture object; incubating the first capture objects and the first antigen molecules for a sufficient time to allow binding of the plurality of first capture objects to the first antigen molecules contained in the sample, thereby creating a first capture object-antigen molecule complex; contacting the first capture object-antigen molecule complex from the previous step with a plurality of first detection probes, wherein the first detection probe binds to the first antigen molecule within the first capture object-antigen molecule complex; labeling the first product with a first detectable moiety; and detecting the first detectable moiety; and (2) contacting the subject sample containing the second antigen molecules with a plurality of second capture objects, wherein each second capture object comprises a second capture probe coupled to a plurality of second capture moieties that specifically bind to the second antigen molecules expressed by M. tb, wherein the subject sample and the plurality of second capture objects are contacted at a ratio that allows for a single second antigen molecule to bind to a single second capture object; incubating the second capture objects and the second antigen molecules for a sufficient time to allow binding of the plurality of second capture objects to the second antigen molecules contained in the sample, thereby creating a second capture object-antigen molecule complex; contacting the second capture object-antigen molecule complex from the previous step with a plurality of second detection probes, wherein the second detection probe binds to the second antigen molecule within the second capture object-antigen molecule complex; labeling the second product with a second detectable moiety; and detecting the second detectable moiety.

In some embodiments, the methods further comprise quantifying the concentration of the first antigen and the second antigen in the subject sample by determining the number of the first detected moieties and the second detected moieties, and comparing the concentration of the first antigen and the second antigen with a control value determined using a sample from a healthy subject, wherein a concentration of the first antigen and the second antigen greater than the control value indicates the presence of Mycobacterium tuberculosis infection in the subject and a concentration of the first antigen and the second antigen less than the control value indicates the absence of Mycobacterium tuberculosis infection in the subject, thereby diagnosing the Mycobacterium tuberculosis infection in the subject.

In one embodiment, the first detectable moiety and the second detectable moiety are the same. In another embodiment, the first detectable moiety and the second detectable moiety are different.

The methods described herein can be used to detect any antigen expressed by M. tb. Exemplary M. tb antigens include, but are not limited to, lipoarabinomannan (LAM) and Ag85B (Rv1886c).

The term “lipoarabinomannan,” also known as “LAM,” is a lipoglycan and major virulence factor specifically produced by Mycobacterium tuberculosis. In addition to serving as a major cell wall component, it is thought to serve as a modulin with immunoregulatory and anti-inflammatory effects. This allows the bacterium to maintain survival in the human reservoir by undermining host resistance and acquired immune responses (Guérardel, Yann; et al. (2003). Lipomannan and Lipoarabinomannan from a Clinical Isolate of Mycobacterium kansasii. Journal of Biological Chemistry. 278 (38): 36637-36651.) LAM is released from metabolically active and degrading mycobacteria and excreted through urine filtration, and is abundantly in the in vitro culture of M. tuberculosis with evidences supporting LAM being actively secreted from infected alveolar macrophage (Wood R, et al. (2012). Lipoarabinomannan in urine during tuberculosis treatment: association with host and pathogen factors and mycobacteriuria. BMC Infect Dis. 12:47).

The term “Ag85B” refers to a member of the antigen 85 (Ag85) complex, which consists of a family of three closely related proteins, Ag85A, Ag85B and Ag85C. The Ag85 complex is the most abundant protein in M. tuberculosis culture supernatants. All three of these proteins exhibit enzymatic activity as mycolyl transferases, in which they catalyze transesterification reactions to synthesize trehalose monomycolate (TMM), trehalose dimycolate (TDM), and mycolated arabinogalactan (Wiker H G, Harboe M. (1992). The antigen 85 complex: a major secretion product of Mycobacterium tuberculosis. Microbiol Rev 56:648-661; Backus K M, et al. (2014). The three Mycobacterium tuberculosis antigen 85 isoforms have unique substrates and activities determined by non-active site regions. J Biol Chem 289:25041-25053).

In some embodiments, the antigen molecule expressed by Mycobacterium tuberculosis is one or more antigens selected from the group consisting of lipoarabinomannan (LAM) and Ag85B (Rv1886c). In one embodiment, the antigen expressed by Mycobacterium tuberculosis is LAM. In another embodiment, the antigen expressed by Mycobacterium tuberculosis is Ag85B. In yet another embodiment, the first and second antigens expressed by Mycobacterium tuberculosis are a combination of LAM and Ag85B.

The capture moieties on the capture objects specifically recognize and bind to an epitope on an M. tb antigen. In some embodiments, the methods of the invention can be used to detect a single antigen molecule. In some embodiments, the methods of the invention can be used to detect multiple antigen molecules. In some embodiments, different epitopes on the same antigen molecule can be detected simultaneously by different groups of capture objects.

The antigen molecules are contacted and captured by a plurality of capture objects. Each capture object comprises a capture probe coupled to a plurality of capture moieties that specifically bind to an M. tb antigen molecule.

The capture probe in the capture object supports and is coupled to a plurality of capture moieties (e.g., anti-M. tb antibodies) that specifically bind to the M. tb antigen. Any suitable capture probe can be used in the context of the invention, including, without limitation, beads (e.g., paramagnetic beads), nanotubes, polymers, plates, disks, dipsticks, or the like. Suitable beads include, but are not limited to, paramagnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads.

In some embodiments, the capture moiety is covalently or non-covalently coupled to the capture probe (e.g., paramagnetic bead) to form the capture object. The capture moieties may be covalently linked to the bead using any suitable conjugation approach known in the art or described herein. The immobilized capture moieties may be non-covalently linked to the capture probes, for example, by forming a non-covalent affinity binding pair.

The plurality of capture moieties specifically bind to the M. tb antigen molecule. In some embodiments, the capture moiety is an antibody. In some embodiments, the capture moiety is an anti-M tb antibody. The anti-M tb antibody is obtained from a human, a recombinant bacterium, a non-human, non-primate mammal such as a rabbit, a mouse, a rat, a sheep, a goat, or other animal. In some anti-M tb antibody is a rabbit IgG molecule specific to an antigen expressed by M. tb. In some embodiments, the capture moiety is a full-length antibody, an antigen-binding fragment of an antibody, or an antibody mimetic. In some embodiments, the full-length antibody is an IgG, IgA, IgD, IgE, or IgM antibody. In some embodiments, the antigen-binding fragment is an scFv, an Fv, a dAb, a Fab, an Fab′, an Fab′2, an F(ab′)2, an Fd, an Fv, or an Feb. In some embodiments, the antibody mimetic is an affibody, an affilin, an affimer, an affitin, an alphabody, an anticalin, an avimer, a DARPin, a fynomer, a Kunitz domain peptide, a monobody, or a nanoCLAMP.

The capture object may be detectably labeled to allow for detection of different epitopes on the same antigen. For example, in multiplexed assays as described in the Examples herein, a first population of capture objects may be detectably labeled with a first label, and a second population of capture objects may be detectably labeled with a second label, such that the first population and the second population are distinguishable (also referred to herein as “distinguishably labeled”). In some embodiments, the capture probe in the capture object is detectably labeled. In some embodiments, the capture moiety in the capture object is detectably labeled.

Any suitable label can be used. For example, the label may be a reporter dye (e.g., a fluorescent dye, a chromophore, or a phospho), or a mixture thereof). By varying both the composition of the mixture (i.e. the ratio of one dye to another) and the concentration of the dye (leading to differences in signal intensity), matrices of unique tags may be generated. Capture probes (e.g., beads) can be labeled using any suitable approach, for example, by covalently attaching the label (e.g., a dye) to the surface of the capture probes, or alternatively, by entrapping the label (e.g., a dye) within the capture object. Such dyes may be, for example, covalently attached to the surface of a capture probe (e.g., a bead), for example, using any of the conjugation approaches described above or herein. Suitable dyes for use in the invention include, but are not limited to, ALEXA FLUOR® dyes, CY® dyes, DYLIGHT® dyes, fluorescein, rhodamine, tetramethylrhodamine, eosin, erythrosin, coumarin, methyl-coumarins, pyrene, Malachite green, fluorescent lanthanide complexes, including those of europium 5 and terbium, stilbene, Lucifer Yellow, CASCADE BLUE™ TEXAS RED®, and others known in the art (e.g., as described in The Molecular Probes Handbook, 11th Ed., 2010).

The total number of capture moieties (e.g., antibody molecules) coupled to a capture probe (e.g., bead) on a single capture object may be between about 1 and about 10,000,000, between about 50,000 and about 5,000,000, or between about 100,000 and about 1,000,000. In some embodiments, the total number of capture objects provided is at least about 10,000, at least about 50,000, at least about 100,000, at least about 1,000,000, at least about 5,000,000, at least about 10,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, or at least about 5,000,000,000.

The total number of capture objects provided for contacting the subject sample containing a target molecule may be between about 1,000 to about 5,000,000 capture objects, e.g., about 1000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, about 1,000,000, about 2,000,000, about 3,000,000, about 4,000,000, or about 5,000,000 capture objects. In some embodiments, any of the methods disclosed herein may involve contacting the sample with about 10,000 to about 5,000,000 capture objects, about 10,000 to about 4,000,000 capture objects, about 10,000 to about 3,000,000 capture objects, about 10,000 to about 2,000,000 capture objects, about 10,000 to about 1,000,000 capture objects, about 10,000 to about 500,000 capture objects, about 10,000 to about 400,000 capture objects, about 10,000 to about 300,000 capture objects, about 10,000 to about 200,000 capture objects, or about 10,000 to about 100,000 capture objects.

Contacting the subject sample containing the target molecule (M. tb antigen molecule) with the capture object is performed for a duration of time and under conditions favorable to binding of the M. tb antigen molecule with the capture moiety, such as the capture antibody, to the antigen.

Prior to contacting with the capture objects, the sample is subject to certain treatments. For example, the sample is subject to centrifugation prior to contacting with the capture objects. Alternatively, prior to contacting with the capture objects, the sample is subject to heat treatment followed by centrifugation; or the sample is subject to centrifugation followed by heat treatment.

The capture objects and the subject sample containing the target molecule (e.g., the M. tb antigen) are incubated to allow binding of the capture objects to the target molecule. In some embodiments, the incubation can be performed for less than about 1 minute; about 1 minute to about 8 hours, e.g., about 1 minute about 5 minutes, about 10 minutes, about 20 minutes, about 30 minutes, about 40 minutes, about 50 minutes, about 60 minutes, about 2 hours, about 3 hours, about 4 hours, about 5 hours, about 6 hours, about 7 hours, or about 8 hours. In some embodiments, the incubation of the capture object with the subject sample containing the M. tb antigen is about 15 minutes to about 35 minutes.

The capture objects and the subject sample containing the M. tb antigen may be incubated under a physiological condition that maintain the secondary and tertiary protein structure and the conformations of the epitopes on antigen molecules. In some embodiments, the incubation is performed under a pH of about 5, about 6, about 7, or about 8. In some embodiments, the incubation is performed at a temperature of about 4° C. to about 37° C. In some embodiments, the incubation is performed at about 4° C. In some embodiments, the incubation is performed at about 25° C. In some embodiments, the incubation is performed at about 37° C.

To allow for single molecule detection, the capture objects are incubated with the target molecule (e.g., the M. tb antigen molecule) at a ratio that allows for a single target molecule to bind to a single capture object. In particular, the amount of the capture objects may exceed the amount of the M. tb antigen molecules contained in the subject sample to allow for a distribution of the target molecule on the capture object that at least some of the capture objects bind to M. tb antigen molecules and a portion of the capture objects do not bind with any antigen molecule. For example, the ratio of capture object and target molecule can be about 1:1 to about 50:1. In some embodiments, the ratio of capture object and target molecule is about 1:1, about 2:1, about 3:1, about 4:1, about 5:1, about 10:1, about 15:1, about 20:1, about 25:1, about 30:1, about 35:1, about 40:1, about 45:1, about 50:1. In some embodiments, the ratio between capture objects and the antigen molecules in the incubation is about 50:1.

In some embodiments, the plurality of capture objects and the antigens are incubated at a ratio of about 1:100,000 to about 50:1. In one embodiment, the plurality of capture objects and the antigens are incubated at a ratio of about 1:90,000, about 1:80,000, about 1:70,000, about 1:60,000, about 1:50,000, about 1:40,000, about 1:30,000, about 1:20,000, about 1:10,000, about 1:9000, about 1:8000; about 1:7000, about 1:6000, about 1:5000, about 1:4000; about 1:3000; about 1:2000, about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, or about 1:5. In one embodiment, the plurality of capture objects and the antigens are incubated at a ratio of about 1:100,000. In one embodiment, the plurality of capture objects and the antigens are incubated at a ratio of about 1:30.

In some embodiments, at least some of the capture objects bind to antigen molecules and a portion of the capture objects do not bind with any antigen molecule. In some embodiments, at least 10%, 20%, 30%, 40%, 50%, 60%, 70%, 80%, or 90% of the capture objects are not bound to any antigen molecule. In preferred embodiments, at least 80% of the capture objects are not bound to any antigen molecule.

Upon contacting and incubation, the capture objects and the M. tb antigen molecule form capture object-antigen molecule complexes. In some embodiments, at least 90%, 91%, 92%, 93%, 94%, 95%, 96%, 97%, 98%, 99% of the object-antigen molecule complexes have a single M. tb antigen molecule bound to a single capture moiety on each capture object.

The methods disclosed herein provides an advantageously high dynamic range of detecting and quantifying antigen molecules in a subject sample. In some embodiments, the ratio of capture object and target molecule can be adjusted to allow more than one antigen molecules to bind to a capture object, and the antigen molecules are detected and quantified according to the intensity of signals generated in the later steps described herein.

In some embodiments, the plurality of capture objects and the antigens are incubated at a ratio of about 1:100,000 to about 50:1. In one embodiment, the plurality of capture objects and the antigens are incubated at a ratio of about 1:90,000, about 1:80,000, about 1:70,000, about 1:60,000, about 1:50,000, about 1:40,000, about 1:30,000, about 1:20,000, about 1:10,000, about 1:9000, about 1:8000; about 1:7000, about 1:6000, about 1:5000, about 1:4000; about 1:3000; about 1:2000, about 1:1000, about 1:900, about 1:800, about 1:700, about 1:600, about 1:500, about 1:400, about 1:300, about 1:200, about 1:100, about 1:90, about 1:80, about 1:70, about 1:60, about 1:50, about 1:40, about 1:30, about 1:20, about 1:10, or about 1:5. In one embodiment, the plurality of capture objects and the antigens are incubated at a ratio of about 1:100,000. In one embodiment, the plurality of capture objects and the antigens are incubated at a ratio of about 1:30.

In one embodiment, at least 70% of the capture objects are bound to one or more antigens.

In some embodiments, more than one group of capture objects are incubated with a subject sample containing the target molecule (e.g., the M. tb antigen molecule). For example, different capture objects can be designed so that they specifically bind to different epitopes on each of the same antigen molecules. In other embodiments, different capture objects can be designed to bind different target molecules. By detecting different epitopes simultaneously on the same antigen, or by detecting a combination of target molecules, (e.g., the M. tb antigen molecules, e.g., Ag85B and LAM), the methods of the invention have significant advantages in accuracy and sensitivity, compared to traditional assays.

In some embodiments, the capture objects that specifically bind to a first epitope on an antigen are distinguishably labeled from capture objects that specifically bind to a second epitope on an antigen so that different labels can be distinguished. In some embodiments, more than one group of distinguishably labeled capture objects are incubated with the subject sample containing the target molecule (e.g., the M. tb antigen molecule), and each group of capture objects specifically bind to one distinct epitope on an antigen molecule expressed by M. tb. In some embodiments, each group of distinguishably labeled capture objects are distinguishably detected, therefore distinct epitopes on the same antigen are distinguishably detected. In some embodiments, three groups of capture objects are incubated with the subject sample containing the target molecule (e.g., the M. tb antigen molecule).

In other embodiments, different capture objects can be designed to bind different target molecules, e.g., a combination of target molecules, (e.g., the M. tb antigen molecules, e.g., Ag85B and LAM). In some embodiments, the capture objects that specifically bind to a first antigen are distinguishably labeled from capture objects that specifically bind to a second antigen so that different labels can be distinguished. In some embodiments, more than one group of distinguishably labeled capture objects are incubated with the subject sample containing the target molecules (e.g., a combination of target molecules, e.g., the M. tb antigen molecules, e.g., Ag85B and LAM), and each group of capture objects specifically bind to one distinct antigen molecule expressed by M. tb.

The methods may further comprise a washing step after the incubation of capture objects with the subject sample containing the target molecule (e.g., the M. tb antigen molecule). The washing step removes excess subject sample and unbound M. tb antigen molecules. In some embodiments, the capture objects are washed with a buffered solution. In some embodiments, the buffered solution is phosphate-buffered saline (PBS).

Following the washing step, the capture object-antigen molecule complex is contacted with a plurality of detection probes (e.g., detection antibodies).

A detection probe, such as a detection antibody, specifically binds to an M. tb antigen molecule, and is linked to a first member of an affinity binding pair, wherein the detectable moiety is linked to the second member of the affinity binding pair.

Any suitable detection probe may be used in the context of the present invention. For example, in some embodiments, the detection probe is an antibody (e.g., a full-length antibody (e.g., an IgG, IgA, IgD, IgE, or IgM antibody) or an antigen-binding antibody fragment (e.g., an scFv, an Fv, a dAb, a Fab, anFab′, an Fab′2, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule. In some embodiments, the detection probe is covalently or non-covalently linked to a detectable moiety. In some embodiments, the non-covalent affinity binding pair is biotin-streptavidin, biotin-avidin, ligand-receptor, antigen-antibody, or antibody binding protein antibody. In some embodiments, the non-covalent affinity binding pair is biotin-streptavidin. In some embodiments, the detection probe is biotinylated. In some embodiments, the detection probe is biotinylated and the detectable moiety is conjugated with a streptavidin.

A detection antibody is contacted with the capture object-target molecule complex (wherein the capture object is bound to the M. tb antigen). In some embodiments, for direct detection of non-complexed M. tb antigens in the sample, the detection antibody comprises a biotinylated anti-M. tb antibody.

The methods of the invention may further comprise labeling the detection probe-bound, capture object-target molecule complex (wherein the capture object is bound to the M. tb antigen) with a detectable moiety. The detectable moiety is linked to a second member of an affinity binding pair and can be linked to the detection probe-bound, capture object-target molecule complex via the affinity binding pair.

In some embodiments, the detectable moiety is, or comprises an enzymatic label (e.g., beta-galactosidase, horseradish peroxidase, glucose oxidase, and alkaline phosphatase), a fluorescent label, a radioactive label, or a metal label. For example, in some embodiments, an enzymatic label generates a species (for example, a fluorescent product) that is either directly or indirectly detectable optically. In some embodiments, the method includes detecting a product of an enzymatic reaction as an indication of the presence of the enzymatic label. In some embodiments, the product of the enzymatic reaction is detected upon its release from the enzymatic label in a zone around the discrete site where the enzyme and/or antigen (or antigen-antibody complex, depending on the embodiment) is located (e.g., in a microwell on an array as described herein, e.g., a Simoa® array). In some embodiments, the detectable label on a detectable moiety is β-galactosidase, the enzyme substrate added to the array can be a β-galactosidase substrate such as resorufin-β-D-galactopyranoside or fluorescein di(β-dgalactopyranoside). Additional examples include a variety of enzymatic labels or colored labels (for example, metallic nanoparticles (e.g., gold nanoparticles), semiconductor nanoparticles, semiconductor nanocrystals (e.g., quantum dots), spectroscopic labels (for example, fluorescent labels), and radioactive labels) may be used in the methods described herein. The presence of the detectable moiety can be detected using suitable detection systems, for example, optical detectors (for example, intensified CCD cameras), or any other suitable detectors known in the art.

The methods of the invention further include determining the concentration of the M. tb antigen molecule, for example, by determining the number of detectable moieties bound to the capture object complexes from the previous steps.

The number of the detectable moieties may be determined by any ultrasensitive detection method, e.g., a bead-based technology, for example, using the ultrasensitive Single Molecule Array (Simoa) technology. Simoa is a registered trademark owned by the Quanterix Corporation (Billerica, Mass., U.S.A.). As defined herein, Simoa platform offers a 50 to 1000-fold increase in sensitivity for protein detection, compared to ELISA, utilizing a bead-based immunoassay format with multiplex ability that is amenable to implementation in future POC devices. Unlike conventional ELISA methods, Simoa traps single bead-bound molecules in femtoliter-sized wells and provides a “digital” readout of each individual bead to determine whether the bead is bound to the target molecule. The digital nature (e.g., “yes-no” or “true-false”) of the result for each well creates an average increase in sensitivity of three orders of magnitude—1,000 times—over conventional ELISA with coefficients of variation (CV) less than about ten percent.

In some embodiments, a detecting step includes single-molecule detection of the detectable moieties. The detection step can be performed by any ultrasensitive detection method. For example, in some embodiments, a detection step occurs in an array of microwells, wherein the microwells are capable of holding zero or one capture object. For example, the methods may involve placing the mixture from the previous steps in a plurality of reaction vessels (e.g., microwells). The array may be as described herein or as described, for example, in WO 2014/183096; WO 2010/039179, WO 2009/029073; US 2018/0136203; US 2018/0017552; US 2018/0003703; US 2015/0355182; US 2015/0353997; US 2010/0075355; US 2010/0075439; U.S. Pat. No. 8,846,415; or U.S. Pat. No. 9,482,662, which are incorporated herein by reference in their entirety. In some embodiments, the array is a Quanterix® single molecule array (e.g., Simoa®.)

In some embodiments, the methods described herein may utilize a plurality or an array of reaction vessels (e.g., microwells) to determine the presence or concentration of one or more target molecules (e.g., M. tb antigen molecules). An array of reaction vessels allows a fluid sample to be partitioned into a plurality of discrete reaction volumes during one or more steps of a method. In some embodiments, the reaction vessels may all have approximately the same volume. In other embodiments, the reaction vessels may have differing volumes.

The reaction vessels (e.g., microwells) may have any suitable volume. The microwells may have a volume of about 10 attoliter (aL) to about 10 picoliters (pL). In some embodiments, the microwells have a volume of about 40 femtoliters. In some embodiments, the microwells have a volume of about 10 femtoliters-1 picoliter. In some embodiments, the microwells have a volume of about 10 femtoliters-500 femtoliters. In some embodiments, the microwells have a volume of about 30-100 femtoliters.

For embodiments employing an array of reaction vessels (e.g., microwells), any suitable number of reaction vessels (e.g., microwells) can be used. Arrays containing from about 2 to many billions of reaction vessels can be made by utilizing a variety of techniques and materials. Increasing the number of reaction vessels in the array can be used to increase the dynamic range of an assay or to allow multiple samples or multiple types of molecules to be assayed in parallel. For example, to allow simultaneous detection and quantification of multiple epitopes of the same antigen molecule, and simultaneous detection and quantification of multiple types of M. tb antigen molecules. In some embodiments, the array comprises between one thousand and one million reaction vessels per sample to be analyzed.

In some cases, the array comprises greater than one million reaction vessels. In some embodiments, the array comprises between about 1,000 and about 50,000, between about 1,000 and about 1,000,000, between about 1,000 and about 10,000, between about 10,000 and about 100,000, between about 100,000 and about 1,000,000, between about 1,000 and about 100,000, between about 50,000 and about 100,000, between about 20,000 and about 80,000, between about 30,000 and about 70,000, between about 40,000 and about 60,000, or about 50,000, reaction vessels.

Individual reaction vessels may contain a binding surface. The binding surface may comprise essentially the entirety or only a portion of the interior surface of the reaction vessel or may be on the surface of another material or object that is confined within the reaction vessel, such as, for example, the complex of the capture object and the M. tb antigen molecule. For example, in some embodiments, a reaction vessel may be any reaction vessel described in WO 2009/029073, which is incorporated herein by reference in its entirety.

The methods may further include sealing the microwells. At least some of the capture objects (e.g., at least some associated with at least one target antigen) may be spatially separated/segregated into a plurality of locations, and at least some of the locations may be addressed/interrogated. A measure of the concentration of M. tb antigen molecules in the sample may be determined based on the information received when addressing the locations.

For example, in some cases, a measure of the concentration may be based at least in part on the number of locations (e.g., microwells) determined to contain a capture object that is or was associated with at least one detectable moiety. In some embodiments, such as digital immunoassays for detection of an M. tb antigen as described herein, the number of locations determined to contain a capture object that is or was associated with at least one detectable moiety may be related to the concentration of the target molecule in the sample. In other cases and/or under differing conditions, a measure of the concentration may be based at least in part on an intensity level of at least one signal indicative of the presence of a plurality of target molecules (e.g., M. tb antigen molecules) and/or capture objects associated with a target molecule (e.g., M. tb antigen molecules) at one or more of the addressed locations. In some embodiments, the number/fraction of locations containing a capture object but not containing a detectable moiety or a target molecule (e.g., M. tb antigen molecules) may also be determined and/or the number/fraction of locations not containing any capture object may also be determined.

In some embodiments, multiple groups of distinguishably labeled capture objects or detectable moieties in complex with the capture objects may be detected simultaneously. Simultaneous addressing/detection can be accomplished by using various techniques, including optical techniques (e.g., using a charge coupled device (CCD) detector, charge-injection device (CID), or complementary-metal-oxide-semiconductor detector (CMOS) detector). Any suitable detector may be used in the methods described herein.

In some embodiments, the detection of step (e) comprises detecting one or more antigens bound to the plurality of capture objects.

In some embodiments, multiple detectable moieties on the detection probes bound to the one or more antigens are detected on a single capture object.

In some embodiments, the multiple detectable moieties are detected by the intensity of the fluorescent signal of each of the capture objects.

In some embodiments, quantification of an intensity of the detectable moiety is proportional to a concentration of the M. tb antigen molecule in the sample. In some embodiments, the concentration is extrapolated from a calibration curve generated by applying the methods herein to samples having known concentrations of the capture antigen or samples from healthy subjects. Comparing the intensity of the detectable labels, such a fluorescence, to the intensity generated by control samples may be used to distinguish between two different disease states.

Therefore, the methods of the invention provide highly sensitive and accurate measurement of concentrations of M. tb antigen. The methods allow for the detection of target molecules on the level of a single target molecule (e.g., M. tb antigen).

In some embodiments, the concentration of the M. tb antigen molecule is from about 0 to about 100 nanograms/milliliter (ng/mL). In some embodiments, the concentration of the M. tb antigen molecule is about 0 to about 20 ng/mL. In some embodiments, the concentration of the M. tb antigen molecule is about 0 to about 0.2 ng/mL. In some embodiments, the concentration of the M. tb antigen molecule is about 0 to about 100 picograms/milliliter (pg/mL). In some embodiments, the concentration of the M. tb antigen molecule is about 0 to about 10 pg/mL. In some embodiments, the concentration of the M. tb antigen molecule is about 0 to about 1 pg/mL. In some embodiments, the concentration of the M. tb antigen molecule is about 0 to about 0.1 pg/mL.

The methods of the invention may further include diagnosing Mycobacterium tuberculosis infection in a subject suspected of having M. tb infection, for example, by comparing the concentration of M. tb antigen molecule determined using the methods of the invention with a control value.

The term “standard level” or “control value” refers to an accepted or pre-determined level of an M. tb antigen molecule (which may or may not be present, depending on the source of the control). A control value is used to compare the level of the M. tb antigen molecule in a sample derived from a subject. In some embodiments, the control value of an M. tb antigen molecule is based on the level of the M. tb antigen molecule in a sample(s) from a subject(s) who is not infected with TB. In some embodiments, the control value of an M. tb antigen molecule is based on the level of the M. tb antigen molecule in a sample(s) from a subject(s) who has been exposed to TB, but is asymptomatic (does not present any TB symptoms). In some embodiments, the control value of an M. tb antigen molecule is based on the level of the M. tb antigen molecule in a sample(s) from a subject(s) having active TB. In some embodiments, the control value of an M. tb antigen molecule is based on the level of the M. tb antigen molecule in a sample(s) from a subject(s) having latent TB. In some embodiments, the control value of an M. tb antigen molecule in a sample from a subject is a level of the M. tb antigen molecule previously determined in a sample(s) from the subject. In yet other embodiments, the control level of an M. tb antigen molecule is based on the level of the M. tb antigen molecule in a sample from a subject(s) prior to the administration of a therapy for TB. In some embodiments, the control level of an M. tb antigen molecule is based on the expression level of the M. tb antigen molecule in a sample(s) from an animal model of TB, a cell, or a cell line derived from the animal model of TB.

Alternatively, and particularly as further information becomes available as a result of routine performance of the methods described herein, population-average values for “control” level of expression of an M. tb antigen molecule may be used. In other embodiments, the “control” level of an M. tb antigen molecule may be determined by determining the level of an M. tb antigen molecule in a subject sample obtained from a subject before the onset of latent TB, from archived subject samples, and the like.

A “level of an M. tb antigen molecule” or “the concentration of an M. tb antigen molecule” refers to an amount of an M. tb antigen molecule present in a sample being tested. A level of an M. tb antigen molecule may be either in absolute level or amount (e.g., pg/mL) or a relative level or amount (e.g., relative intensity of signals).

A “greater value”, “greater concentration”, or an “increase in the level” of M. tb antigen molecule refers to a level of an M. tb antigen molecule in a test sample that is greater than the standard error of the assay employed to assess the level of the M. tb antigen molecule, and is preferably at least twice, and more preferably three, four, five, six, seven, eight, nine, or ten or more times the level of the M. tb antigen molecule in a control sample (e.g., a sample from a subject who is not infected with TB, a subject who has been exposed to TB but is asymptomatic, a subject having active TB, and/or the average level of the marker in several control samples).

A “lower value”, “lower concentration”, or a “decrease in the level” of M. tb antigen molecule refers to a level of an M. tb antigen molecule in a test sample that is lower than the standard error of the assay employed to assess the level of the M. tb antigen molecule, and is preferably at least twice, and more preferably three, four, five, six, seven, eight, nine, or ten or more folds lower than the level of the M. tb antigen molecule in a control sample (e.g., a sample from a subject who is not infected with TB, a subject who has been exposed to TB but is asymptomatic, a subject having active TB, and/or the average level of the marker in several control samples).

In some embodiments, an M. tb antigen molecule concentration greater than the control value indicates the presence of M. tb infection in the subject and an antigen molecule concentration less than the control value indicates the absence of M. tb infection in the subject. In some embodiments, the M. tb infection is an active M. tb infection. In some embodiments, the M. tb infection is a latent M. tb infection.

In some embodiments, more than one group of capture objects comprising capture moieties that bind to multiple epitopes on an M. tb antigen molecule is used in the methods disclosed herein. Simultaneous detection and quantification of the multiple epitopes on the antigen molecule may significantly increase the accuracy and sensitivity of the described methods.

The methods of the invention may further include determining the HIV status (e.g., HIV positive or HIV negative) of a subject. In some embodiments, the HIV status is used in combination with the levels of the target molecules (e.g., the M. tb antigen molecules, e.g., Ag85B and LAM) detected in the subject sample in order to determine whether a subject is infected with Mycobacterium tuberculosis.

The methods of the invention further include methods of treating an infection with Mycobacterium tuberculosis in a subject diagnosed of having an M. tb infection, for example, by administering an anti-M. tb treatment to the subject diagnosed with an M. tb infection. In some embodiments, the treatment is a drug treatment (e.g., an antibiotic treatment).

Treatment for subjects having TB (e.g., active TB, latent TB, or multi-drug resistant TB) are known in the art. For example, suitable treatment for subjects having active TB include, for example, multi-drug therapies with one or more drugs selected from the group consisting of antimycobacterial rifapentine, isoniazid, rifampin, pyrazinamide, ethambutol, and streptomycin. In some embodiments, the drug treatment is combined with baseline and periodic serum uric acid assessments, and/or visual acuity and red-green color perception testing. The duration of the treatment can be from about 1 week to about 12 months, for example, from about 2 months to about 6 months.

Suitable treatment for subjects having latent TB include, for example, multi-drug therapies with one or more drugs selected from the group consisting of antimycobacterial rifapentine, isoniazid, and rifampin. In some embodiments, the treatment is administered in a 12-dose, once-weekly regimen for a duration of about 1 week to about 12 months, for example, from about 2 months to about 6 months.

The foregoing treatments are suitable for treating subjects that are HIV+ or HIV−. In subjects having active TB or latent TB, who are infected with HIV, the dose of the drug treatment may be adjusted. Furthermore, in subjects receiving protease inhibitors as treatment of HIV/AIDS, rifampin is excluded from the treatment of active or latent TB, and rifabutin may be used in place of rifampin.

Some subjects may develop multidrug-resistant TB after the treatment with certain regimens. In such subjects, the regimens can be modified to utilize at least 3-5 previously unused drugs for which there is in vitro susceptibility by testing the drug efficacy with the subject sample. Exemplary treatments include, but are not limited to, an aminoglycoside, such as streptomycin, amikacin, capreomycin, and kanamycin; a fluoroquinolone, such as levofloxacin, and ciprofloxacin; a thiomide, such as ethionamide and prothionamide; a pyrazinamide, an ethambutol, a cycloserine, a terizidone, a para-aminosalicyclic acid, and a diarylquinoline, such as bedaquiline. Other suitable drugs and regimens suitable for treating TB can also be used in the methods described herein.

Any suitable subject sample may be used in the context of the present invention. For example, in some embodiments, the sample is a biological sample. In some embodiments, the sample is a liquid sample. Exemplary liquid samples include, without limitation, body fluids, such as lymph, whole blood (including fresh or frozen), plasma (including fresh or frozen), serum (including fresh or frozen), a blood fraction containing peripheral blood mononuclear cells, urine (including fresh or frozen), saliva, semen, sweat, lacrimal fluid, synovial fluid, cerebrospinal fluid, feces, mucous, vaginal fluid, and spinal fluid. Methods or obtaining tissue biopsies and body fluids from mammals are well known in the art.

The volume of the fluid sample analyzed may potentially be any amount within a wide range of volumes, depending on a number of factors such as, for example, the number of capture objects used/available, the number of detection probes, and the like. As non-limiting examples, the sample volume may be about 0.01 μl, about 0.1 μl, about 1 μl, about 5 μl, about 10 μl, about 100 μl, about 1 ml, about 5 ml, about 10 ml, or the like. In some cases, the volume of the fluid sample is between about 0.01 μl and about 10 ml, between about 0.01 μl and about 1 ml, between about 0.01 μl and about 100 μl, or between about 0.1 μl and about 10 μl. In some embodiments, each sample is tested in duplicates or triplicates. In some embodiments, the sample volume is about 25 μl per replicate. In some embodiments, the sample volume is about 10 μl per replicate. In some embodiments, the sample volume is about 50 μl per replicate. In some embodiments, the sample volume is about 75 μl per replicate. In some embodiments, the sample volume is about 85 μl per replicate.

In some embodiments, the fluid sample may be diluted prior to use in a method described herein. For example, in embodiments where the source of a target molecule is a body fluid (e.g., blood, plasma, or serum), the fluid may be diluted with an appropriate solvent (e.g., a buffer such as PBS buffer.) A fluid sample may be diluted about 1-fold, about 2-fold, about 3-fold, about 4-fold, about 5-fold, about 6-fold, about 10-fold, about 50-fold, about 100-fold, or greater, prior to use. The sample may be added to a solution comprising the plurality of capture objects or detectable moieties, or the plurality of capture objects or detectable moieties may be added directly to or as a solution to the sample.

### III. Kits and Articles of Manufacture

The invention provides kits and articles of manufacture for measuring a concentration of a target analyte (e.g., a small molecule, e.g., LAM and/or Ag85B) in a fluid sample (e.g., a urine sample). The article or kit may include, for example, a plurality of capture probes (e.g., beads, e.g., paramagnetic beads) and/or an array substrate comprising a plurality of reaction vessels. The reaction vessels may be configured to receive and contain the capture probes. The plurality of capture probes (e.g., beads) may have an average diameter between about 0.1 micrometer and about 100 micrometers and the size of the reaction vessels may be selected such that only either zero or one beads is able to be contained in single reaction vessels. In some cases, the average depth of the reaction vessels is between about 1.0 times and about 1.5 times the average diameter of the beads and the average diameter of the reactions vessels is between about 1.0 times and about 1.9 times the average diameter of the beads. The average volume of the plurality of reaction vessels may be between about 10 attoliters and about 100 picoliters, between about 1 femtoliter and about 1 picoliter, or any desired range. The substrate may comprise any number of reaction vessels, for example, between about 1,000 and about 1,000,000 reaction vessels, between about 10,000 and about 100,000 reaction vessels, or between about 100,000 and about 300,000 reaction vessels, or any other desired range. In certain embodiments, the capture probes (e.g., beads) may have an average diameter between about between about 1 micrometer and about 10 micrometers, between about 1 micrometer and about 5 micrometers, or any range of sizes described herein.

The kits and articles of manufacture described herein may be configured for carrying out any of the methods, e.g., methods of diagnosing a Mycobacterium tuberculosis infection in a subject, or assays as described herein.

The plurality of capture probes (e.g., beads) provided may have a variety of properties and parameters, as described herein. For example, the beads may be magnetic. The plurality of beads may comprise a binding surface linked to one or more immobilized target analytes.

In some embodiments, the kit or article may include a detectable moiety that is linked to one or more immobilized target analytes, as described herein.

The plurality of reaction vessels may be formed in any suitable substrate, as described herein or in US 2018/0017552, which is incorporated herein by reference in its entirety. In some embodiments, the plurality of reaction vessels is formed on the end of a fiber optic bundle. The fiber optic bundle may be prepared (e.g., etched) according to methods known to those of ordinary skill in the art and/or methods described in US 2018/0017552. In other embodiments, the plurality of reactions vessels is formed in a plate or similar substantially planar material (e.g., using lithography or other known techniques). Exemplary suitable materials are described herein. The kit may include any of the array substrates or reaction vessels as described herein.

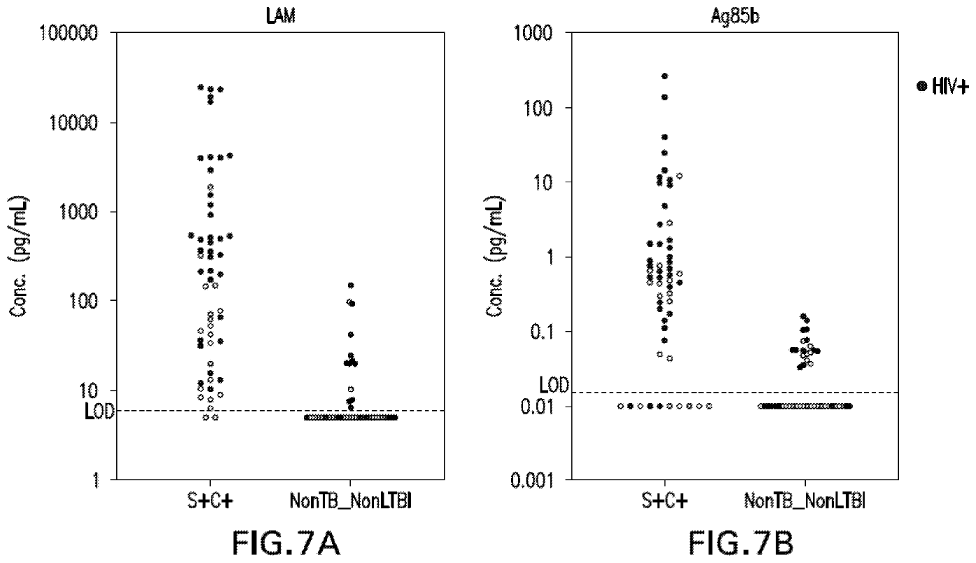

The kit or article may comprise any number of additional components, some of which are described in detail herein. In some cases, the article or kit may further comprise a sealing component configured for sealing the plurality of reaction vessels. In certain embodiments, the plurality of reaction vessels may be formed upon the mating of at least a portion of a sealing component and at least a portion of the second substrate, as shown in FIGS. 7A-7F of US 2018/0017552. As another example, the kit may also provide solutions for carrying out an assay method as described herein. Non-limiting example of solutions include solutions containing one or more types of detection probes, capture probes, or enzymatic label substrates. In some cases, the article or kit may comprise at least one type of control capture probe (e.g., a bead).

In some embodiments, the kit may include instructions for use of components described herein. That is, the kit can include a description of use of the capture probes (e.g., beads) and reaction vessels, for example, for use with a system to determine a measure of the concentration of target analyte(s) in a fluid sample, e.g., a urine sample. As used herein, “instructions” can define a component of instruction and/or promotion, and typically involve written instructions on or associated with packaging of the invention. Instructions also can include any oral or electronic instructions provided in any manner such that a user of the kit will clearly recognize that the instructions are to be associated with the kit. Additionally, the kit may include other components depending on the specific application, as described herein.

This invention is further illustrated by the following examples which should not be construed as limiting. The contents of all references, patents and published patent applications cited throughout this application, as well as the Figures, are hereby incorporated by reference.

### EXAMPLES

The following Materials and Methods were used in the Examples below.

### Materials

Compositions listed herein are by way of working examples only. These example compositions and materials used in performances of specific method steps are not, necessarily, meant to be limiting or to change the scope of the inventions disclosed and taught herein.

EZ-link NHS-PEG4-Biotin, Amicon Ultra-0.5 mL Centrifugal Filters, and 1-Ethyl-3-(3-dimethylaminopropyl) carbodiimide hydrochloride (EDC) were purchased from Thermo Fisher Scientific (Hampton, N.H.). Anti-human IgG monoclonal antibody was purchased from BioLegend (San Diego, Calif.). Phosphate-buffered saline (PBS) and Sigmatrix urine diluent (SAE0074) were purchased from Sigma-Aldrich. Synthetic urine solutions were obtained from Ricca Chemical (Cat #8361-1, Arlington, Tex., USA), and BioIVT (Westbury, N.Y., USA). Newborn calf serum (NBCS) was purchased from Life Technologies. The Simoa HD-1 or HD-X Analyzer, instrument consumables, and Homebrew assay reagents were purchased from Quanterix Corporation (Billerica, Mass.). Instrument consumables include Simoa Discs (Cat #100001), reaction cuvettes (Cat #100803), pipette tips (Cat #101726), wash buffer 1 (Cat #100486), wash buffer 2 (Cat #100487), fluorocarbon oil (Cat #100206), reagent bottles (Cat #102411), 96-well microplates (Cat #101457). Homebrew assay reagents include streptavidin-β-galactosidase (SβG) enzyme concentrate, resorufin-β-D-galactopyranoside (RGP) substrate, buffers (bead wash buffer, bead conjugation buffer, bead blocking buffer, and biotinylation reaction buffer), and diluents (bead diluent, detector diluent, and SOG diluent). Non-encoded carboxyl-functionalized paramagnetic bead, dye-encoded paramagnetic Helper bead labeled as 647L2, and four distinct dye-encoded carboxyl-functionalized paramagnetic bead (2.7 μm in diameter) populations labeled as 700L1.5, 647L1.5, 488L1, and 750L1.5 to identify encoding fluorescence emission wavelengths were also obtained from Quanterix Corporation.

### Preparation of Capture Moiety Coated Beads

The antibody conjugation is similar for both non-encoded beads and dye-encoded beads. The coupling was performed wherein 200 microliters (uL) of dye-encoded carboxyl-modified paramagnetic beads (˜1.2×10⁹ beads per mL) or 100 uL of non-encoded carboxyl-modified paramagnetic beads (˜2.3×10⁹ beads per mL) were washed three times with 600 μL of bead wash buffer (0.1% TWEEN® 20 in 1×PBS, pH 7.4) and twice with bead conjugation buffer. The beads were then suspended in 194 μL bead conjugation buffer. 10 mg/mL EDC was freshly reconstituted in 1 mL bead conjugation buffer and 6 μL was added to the beads and vortex-mixed well. The mixture was incubated for 30 minutes while vigorously shaking at 4° C. After the first incubation, beads were washed once with bead conjugation buffer, and incubated with 200 μL of 0.2 mg/mL capture antibody in bead conjugation buffer for 2 hours while vigorously shaking at 4° C. The beads were then washed twice with bead wash buffer, and final incubation was in bead blocking buffer for 30 minutes at room temperature while vigorously shaking. After incubation, beads were washed once with bead wash buffer, once with bead diluent (Quanterix®), and finally stored in 200 μL of bead diluent at 4° C. until further use. When coupling smaller or larger batches, reaction volume was adjusted proportionally according to the antibody amount.

### Preparation of Biotin-Conjugated Detection Antibodies

Anti-M. tb protein antibodies and anti-human IgG antibody were buffer exchanged to remove the storage buffer by first transferring 0.1 mg of antibody (in solution) to 50K Amicon filter. Biotinylation reaction buffer was added to the filter up to a total volume of 450 μL. The filter device was centrifuged at 14,000×g for 5 minutes followed by discarding the filtrate. This process was repeated twice. The filter was then inverted into a clean collecting tube and centrifuged at 1,000×g for 2 minutes to recover antibody. This process was repeated one more time after rinsing the filter with 50 μL of biotinylation reaction buffer. The antibody concentration was then adjusted to 1 mg/mL in biotinylation reaction buffer. The biotinylation was performed with a 40-fold molar excess of biotin to antibody, and the volume ratio of antibody solution-to-biotin working solution was kept at 97.5%-to-2.5%. First, the required biotin working concentration was calculated. NHS-PEG4-biotin was then reconstituted in water and diluted to desired working concentration. Upon addition of NHS-PEG4-biotin to buffer exchanged antibody, the mixture was incubated at room temperature without shaking for 30 minutes. The biotinylated antibody was purified by performing buffer exchange process one more time with Amicon filter in biotinylation reaction buffer, and the recovered detection antibody was stored at 4° C. until further use.

### Reagent Preparation and HD-1/HD-X Analyzer Operation

The Simoa HD-1 and HD-X Analyzer were used for all Simoa measurements. The instrument was loaded with the reagents, necessary consumables and samples needed to perform the assays. HD-X Analyzer is an upgrade prototype of HD-1 which shares the same consumables and was used to analyze samples from the validation cohort. Reagents, consumables, and samples were loaded onto the instrument. Samples and calibrators were prepared and loaded in 96-well plates. After loading process, the HD-1 Analyzer performed the immunoassay protocol in two-step manner or three-step manner.

The two-step protocol involves a 35 minutes incubation of the capture object and detection antibodies with the calibrators or urine samples, and a 5 minutes SRG incubation; the three-step protocol involves a 15 minutes incubation of the capture probes with the calibrators or urine samples (30 minutes at this step for LAM), a 5 minutes detection antibody incubation, and a 5 minutes SβG incubation. Each incubation step is followed by wash cycles. After incubation, beads are resuspended in RGP, loaded into microarrays, sealed with oil, and imaged. The number of average enzymes per bead (AEB) was calculated, and calibration curves were fit with a four-parameter logistic weighted regression to determine protein concentrations in the samples from previously measured AEB measured values. The assay limits of detection (LODs) were calculated by extrapolating concentrations at signals that are three times the standard deviation (SD) above the assay background.

For M. tb antigen assays, the serial-diluted calibrators were prepared in sample diluent (Quanterix®), except for McelA, which was prepared in 1×PBS buffer with 5% NBCS. Urine samples were diluted 2-fold for LAM in sample diluent A (Quanterix®), or as 75% sample plus 25% sample diluent (Quanterix®) for Ag85B using an on-board dilution protocol provided by the HD-1 Analyzer. After loading process, the HD-1 Analyzer performed the immunoassay protocol in two-step manner (for RelG, McelA, GlcB, HspX, and CFP10 assays) or three-step manner (for Ag85A, Ag85B, Ag85C, Adk, GroES, and LAM).

Specifics of assays for each antigen are as follows: Ag85B, Ag85A, ADK, and Ag85C were developed as 4-plex assay and applied three-step assay manner, with reagent concentrations of 6,000,000 total beads per mL (1,500,000 beads per plex per mL), and 0.3 ug/mL final detection antibody per antigen. Serially diluted calibrators were prepared in the diluent composed of 75% synthetic urine (Ricca Chemical) and 25% sample diluent (Quanterix), and urine samples were diluted as 75% sample plus 25% sample diluent (Quanterix). GlcB and RelG were developed as 2-plex assay in two-step manner, with 5,000,000 beads per mL (2,500,000 beads per plex per mL) and working detector concentration of 0.1 ug/mL for RelG, 0.17 ug/mL for GlcB. Calibrators were prepared in diluent composed of 75% synthetic urine (Ricca Chemical) and 25% sample diluent (Quanterix) and urine samples were diluted as 75% sample plus 25% sample diluent (Quanterix). All other antigen assays were developed as single-plex assays, with bead concentrations of 5,000,000 beads per mL (2,500,000 assay beads plUS 2,500,000 Helper beads per mL). CFP10, McelA, and HspX assays were two-step format, while GroES and LAM assays were three-step format. The final concentrations of detection antibodies were 0.3 ug/mL for GroES, 0.17 ug/mL for CFP10 and McelA, 0.13 ug/mL for HspX, and 0.6 ug/mL for LAM. For GroES and HspX assays, calibrators were prepared in diluent composed of 75% synthetic urine (Ricca Chemical) and 25% sample diluent (Quanterix), CFP10 calibrators in diluent composed of 75% synthetic urine (BioIVT) and 25% sample diluent (Quanterix), McelA calibrators in Sigmatrix urine diluent, and LAM calibrators in sample diluent (Quanterix). Samples were diluted two-fold in sample diluent (Quanterix) for McelA and LAM, as 75% sample plus 25% sample diluent (Quanterix) for CFP10 and HspX, and as 75% sample plus 25% TBST (pH 8.0) for GroES.

Urine samples were centrifuged at 10,000 g for 5 min before plated and loaded into HD-1/HD-X Analyzer. All dilutions were carried out on-board by the instrument.

### Assay Development

For M. tb antigen assays, an optimal antibody pair for each target was identified via a pairwise screening process using the HD-1 Analyzer. Available antibodies were paired as either capture reagents (conjugated to beads) and detection reagents (biotinylated). The antibodies were screened at a high target concentration (1,000 pg/mL to 10,000 pg/mL recombinant protein or purified LAM) and blank (0 pg/mL) in three-step format. The identified optimal pair showed a high signal-to-background ratio, with low background signal. Simoa assays were developed and optimized by adjusting various assay parameters including detection antibody concentration, enzyme concentration, assay format, incubation times, and diluent buffers. Assay validation including dilution linearity and spike and recovery was performed for each assay. To assess linearity, M. tb proteins or LAM were spiked into diluted healthy human urine and then performed serial dilution. The linear fitting coefficient was evaluated. To assess the recovery in the serum sample matrix, diluted healthy human urine samples were spiked with various concentrations of appropriate M. tb proteins or LAM. The percent recovery was evaluated.

### Ethic Statement and Sample Information

This study was exempted by Partners Human Research Committee (Protocol #: 2017P001447). 100 urine samples were obtained from the FIND TB sample repository. FIND collected these samples under IRB/IEC approved studies in participating countries. Obtained subject samples were categorized based on clinical and microbiological assessment, as provided by FIND, into TB cases (n=55; smear positive, culture positive) and non-TB controls (NTB; n=45; smear negative, culture negative). All samples were collected prior to medical intervention. Demographics and clinical characteristics data are summarized in Table 3. All clinical data were anonymized, and barcodes were used to access pertinent information.

### Example 1. Direct Detection of M. tuberculosis Protein, Ag85B in Urine Using Ultrasensitive Simoa Assays

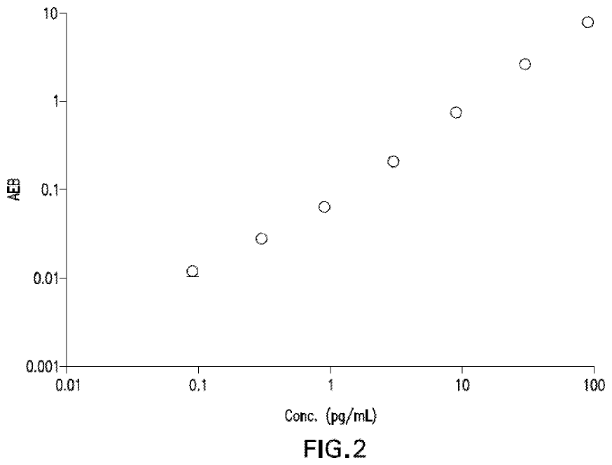

Ultrasensitive Simoa assays were developed for detection of an M. tb antigen, Ag85B, in a urine sample. Monoclonal antibodies acquired from AbCellera were used. FIG. 2 showed the calibration curve for Ag85B assay, with an assay limit of detection (LOD, LOD=Background+(3× Standard Deviation of Background)) of 0.011 pg/mL.

Assay validation tests, including dilution linearity and spike and recovery, were performed to assess accurate sample reading and to determine the appropriate matrix and dilution factor for urine samples. To determine the recovery in the sample urine matrix, recombinant Ag85B protein was spiked into diluted healthy human urine to obtain final concentrations of 1, 4, and 12 pg/mL. Each sample was diluted in Quanterix Sample Diluent buffer at a ratio of 75% sample/25% diluent. Recovery results were shown in Table 1.

TABLE 1
(A) Percent recovery for spike and recovery test for Ag85B; (B) Linear
fitting coefficient of four serial dilutions of spiked samples. CVs of
concentrations after correction for dilution factors are also presented.
(A)

| Sample | Recovery (%) |
|---|---|
| 1 | 101% |
| 2 | 152% |
| 3 | 145% |
| 4 | 117% |
| Average | 129% |

(B)

| | Average | |
|---|---|---|
| | Dilution | |
| | Linearity | Conc. CV |
| Sample | Fitted R² | (corrected) |
| 1 | 0.9723 | 25.2% |
| 2 | 0.999 | 13.6% |
| 3 | 0.9912 | 15.6% |
| 4 | 0.9815 | 21.6% |

### Example 2. Direct Detection of M. tuberculosis Protein, LAM, in Urine Using Ultrasensitive Simoa Assays

Purified M. tb LAM (from culture) and monoclonal antibody CS40 were obtained from BEI Resources. Monoclonal antibodies FIND 24, FIND 28, FIND 29, and FIND 170 were acquired from FIND; S4-20, OTB, and G3 were obtained from Otsuka; A-194 was acquired from the Pinter Lab at Rutgers University.

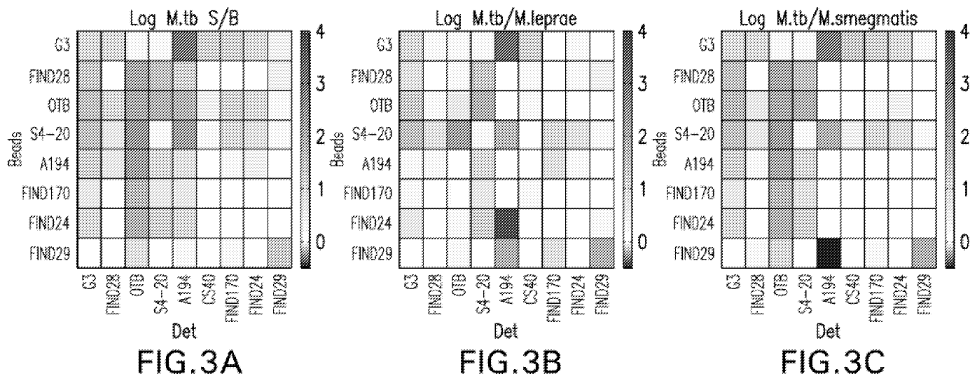

Antibodies were screened on the Simoa platform to identify potential compatible antibody pairs for Simoa assays. Antibody pairs were initially screened at a high target concentration (5,000 pg/mL) and blank (0 pg/mL). Data from antibody pair screening are shown below in FIGS. 3A-C. M. leprae and M. smegmatis LAM were also obtained and screened for antibody pairs against them. A potential candidate pair showed a high signal-to-background (S/B) ratio against M. tb LAM, with low background signal, while providing a high signal ratio of M. tb over non-tb LAM.

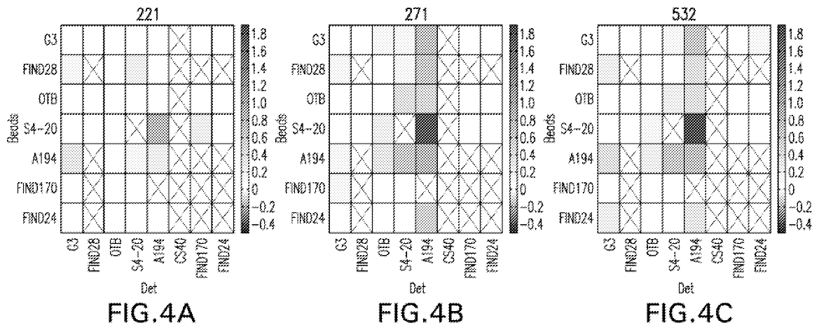

Based on the screening results from purified LAM, several antibody pairs with low S/B ratios, high background readings, or low M. tb over non-tb LAM signal ratios were excluded. For the rest of the pairs, real patient urine samples were screened. Three urine samples were selected from TB+HIV+ individuals, diluted 50-fold and performed the screening process, and the results were shown as below in FIGS. 4A-C.

The screening results suggested that using S4-20 as capture antibody and A194 as detection antibody gave good S/B ratios when screening both purified M. tb LAM and patient samples, as well as good specificities against two demo non-TB LAM. Accordingly, this pair of antibodies was used for assay development and analytical performance validation.

### Sample Pre-Treatment

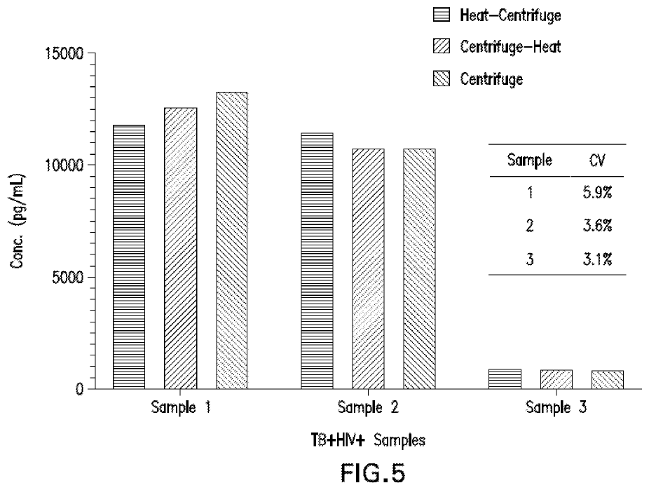

The effect of pre-treatment (centrifuge and heat) on the detectable concentrations of LAM using the chosen antibody pair was subsequently evaluated. Centrifugation, at 10,000 g for 5 minutes, was included as a standard practice in all the assessments in order to minimize matrix interferences from any sediments present in the samples. Heat treatment was also evaluated because of reported dissociating effects on possible complexes present in urine. Samples were evaluated for three conditions: (1) exposed to centrifuging-only, (2) heating before centrifugation and (3) heating after centrifugation. Results were shown in FIG. 5. Within the small tested sample set, the measured concentrations varied only slightly using the different treatment procedures, with a concentration coefficient of variation (CV) of below 6%. Therefore, the heating step was excluded for subsequent assay and only centrifuging was used to test all the samples in order to minimize the necessary pre-treatment steps for future clinical use.

### Assay Analytical Performance Validation

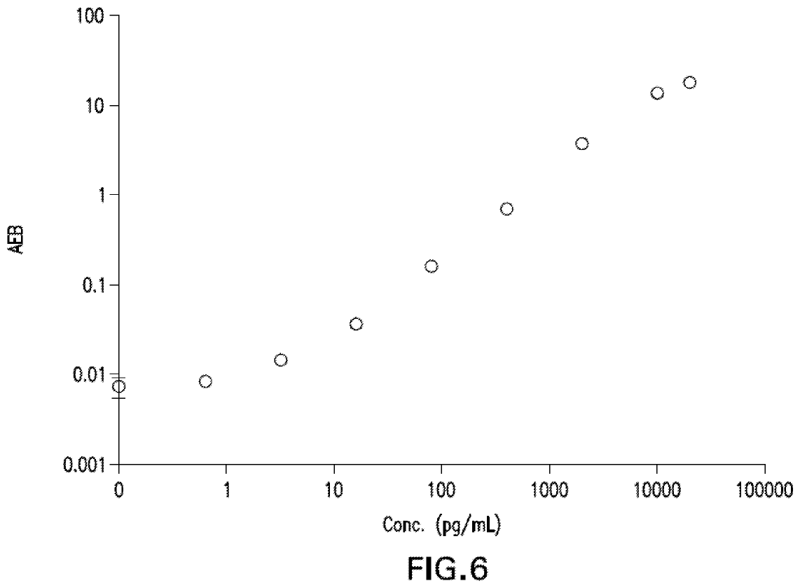

FIG. 6 showed the calibration curve for the final developed LAM assay, with an assay limit of detection (LOD, LOD=Background+(3× Standard Deviation of Background)) of 2.95 pg/mL.

Assay validation tests including spike recovery and dilution linearity were carried out using both TB+ and TB− urine samples. To determine the recovery in the sample urine matrix, various concentrations of the purified LAM were spiked into 2-fold diluted urine samples to obtain final concentrations of 30, 300, and 3,000 pg/mL. Recovery results are shown in Table 2.

TABLE 2
(A) Percent recovery for spike and recovery test for LAM; (B) Linear
fitting coefficient of serial dilutions of spiked samples. Also presented
are CVs of concentrations after correcting for dilution factors.
(A)

| Sample | Recovery (%) |
|---|---|
| TB+HIV+ | 98.7% |
| TB+HIV+ | 96.5% |
| TB−HIV− | 97.0% |
| TB−HIV− | 77.8% |
| TB−HIV− | 78.5% |
| Average | 89.7% |

(B)

| | Average | |
|---|---|---|
| | Dilution | |
| | Linearity | Conc. CV |
| Sample | Fitted R² | (corrected) |
| TB+HIV+ | 0.9606 | 13.6% |
| TB+HIV+ | 0.9818 | 9.2% |
| TB−HIV− | 0.997 | 7.2% |
| TB−HIV− | 0.9942 | 5.4% |
| TB−HIV− | 0.9903 | 6.2% |

### Example 3. Detection in FIND Urine Samples

A total of 100 urine samples were received from FIND, with necessary clinical information available (TB and HIV status). Detailed breakdown of the individual information is provided in Table 3.

TABLE 3
Subject demographics and clinical characteristics

| | | | | All TB+ subjects | |
|---|---|---|---|---|---|
| Number of subjects | | | | (smear+ and | |
| | Total | All TB− subjects | | culture+) | |
| | subjects | HIV− | HIV+ | HIV− | HIV+ |
| Total subjects | 100 | 25 | 20 | 20 | 35 |
| Sex | | | | | |
| Male | 57 | 10 | 6 | 11 | 30 |
| Female | 43 | 15 | 14 | 9 | 5 |
| Geography | | | | | |
| Cambodia | 7 | 7 | 0 | 0 | 0 |
| Peru | 35 | 15 | 0 | 20 | 0 |
| South Africa | 43 | 3 | 20 | 0 | 20 |
| Vietnam | 15 | 0 | 0 | 0 | 15 |
| CD4 count | | | | | |
| ≤100 cells/μL | 23 | 0 | 4 | 0 | 19 |
| >100 cells/μL | 32 | 0 | 16 | 0 | 16 |
| NA | 45 | 25 | 0 | 20 | 0 |

### Assay Performance in Sample Detection

LAM and Ag85B were tested in FIND urine samples. Detectability of each target was summarized in Table 4 and shown in FIGS. 7A-B. Briefly, 53 of 55 (96%) TB+ subjects and 13 of 45 (29%) TB− subjects were found to have LAM concentrations above the sample detection LOD (5.91 pg/mL, corrected from assay LOD for 2-fold dilution of the samples), while 45 of 55 (82%) TB+ subjects and 19/45 (42%) TB− subjects had detectable Ag85B levels (above the LOD of 0.015 pg/mL) in urine.

LAM was detectable in all HIV+ and the large majority of HIV− subjects with TB+ status. The greater detectability of LAM in HIV+TB patients is in agreement with previous studies. A similar pattern was observed for the Ag85B assay, with higher detectability in TB+HIV+ subjects compared to TB+HIV− subjects

TABLE 4
Detectability of LAM and Ag85B in urine samples
Number (%) of subjects

| | All TB− subjects | | | All TB+ subjects | | |
|---|---|---|---|---|---|---|
| | Total | | | Total | | |
| | TB− | | | TB+ | | |
| | subjects | HIV− | HIV+ | subjects | HIV− | HIV+ |
| Total | 45 | 25 | 20 | 55 | 20 | 35 |
| LAM | 13 (29) | 2 (8) | 11 (55) | 53 (96) | 18 (90) | 35 (100) |
| detectable | | | | | | |
| Ag85B | 19 (42) | 7 (28) | 12 (60) | 45 (82) | 13 (65) | 32 (91) |
| detectable | | | | | | |

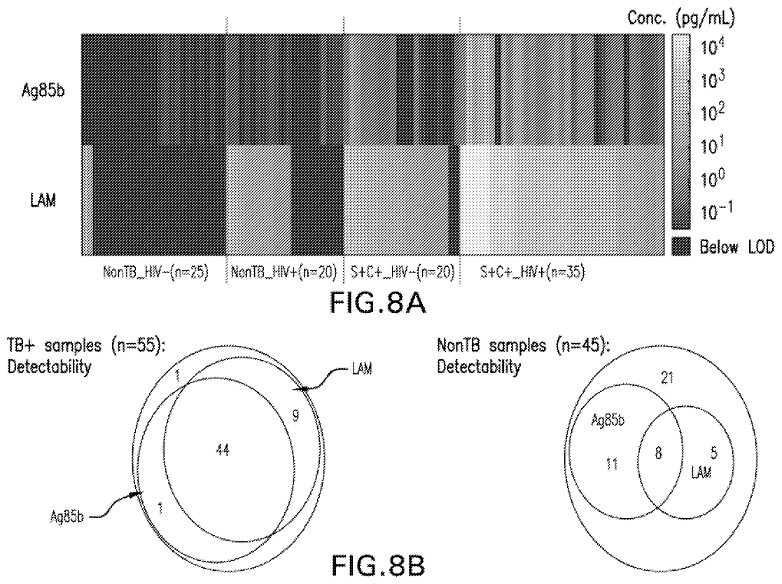

When combining LAM and Ag85B, 54 of 55 (98%) TB+ subjects were found to have at least one detectable target, with 44 (80%) having both of them detectable (FIGS. 8A and 8B). 24 of 45 (53%) TB− subjects had at least one detectable target. Ag85B contributed more to the detectability in the TB− populations. 8 (18%) TB− subjects were found to have both LAM and Ag85B at detectable levels.

### Example 4. Diagnostic Performance Evaluation

Diagnostic performance of LAM-only, Ag85B-only, and LAM/Ag85B combination was evaluated using 10×10 nested stratified cross-validation applied to different models. The models include Random Forest (RF), Support Vector Machine (SVM), Linear Discriminate Analysis (LDA), Quadratic Discriminate Analysis (QDA), Xtreme Gradient Boosting (XGB), and Logistic Regression (LR).

Using only Ag85B as the classifier, the best performance is 0.8717 (S.E. 0.0038) average AUC ROC, achieved by QDA model. The best performance of LAM-only models is 0.9158 (S.E. 0.0036) average AUC ROC, achieved by LR, QDA and LDA. When the two targets are combined, the performance is improved, with the best average AUC ROC of 0.9368 (S.E. 0.0047) achieved by the RF model. The overall average AUC ROC of all the LAM/Ag85B models is 0.9204 (S.E. 0.0025).

When investigating subgroups with different HIV status using the best performing LAM/Ag85B model (RF), the average AUC ROC is 0.9428 (S.E. 0.0032) for HIV+ subjects, and 0.8780 (S.E. 0.0095) for HIV− subjects, providing a better performance of the LAM/Ag85B model in HIV+ population.

Model performance was also evaluated at a fixed sensitivity of 90% in order to develop a triage test. The average specificity is evaluated to be 0.7644 (S.E. 0.0078) for the entire study sample set. With known HIV status as a prior, improvements of specificity are observed. The average specificity at 90% sensitivity is elevated to 0.8400 (S.E. 0.0095) for HIV+ subjects, and 0.8920 (S.E. 0.0196) for HIV− subjects.

### Example 5. Development of Triage Test Based on M. tuberculosis Antigens Using Blinded Urine Samples for Detecting Tuberculosis

### Materials and Reagents

Recombinant CFP10 (NR-49425) and purified M. tb LAM (NR-14848) were both obtained from BEI Resources (Manassas, Va., USA), and HspX was provided by Natural and Medical Sciences Institute at the University of Tuebingen (NMI)) via Foundation for Innovative New Diagnostics (FIND; Geneva, Switzerland) network. LAM was detected using a combination of monoclonal antibody S4-20 (kindly provided by Otsuka Pharmaceutical Co., Tokyo, Japan) as the capture reagent and A194-01 IgM (Rutgers University, Newark, N.J., USA) as the detection reagent. The A194-01 antibody was generated by exchanging the Heavy chain IgG1 constant region with the human IgM constant chain sequence and expressing the antibody in the presence of the human J chain sequence. All the other M. tb recombinant proteins and antibodies were obtained from AbCellera Biologics (Vancouver, Canada) under material transfer agreements for Simoa assay development. After Simoa screening for compatible binding pairs, Genscript was contracted to produce large batches of identified antibodies using sequence information provided by AbCellera Biologics under non-disclosure agreements. General materials and reagents are provided above.

### Simoa Assays

Digital immunoassays were performed using the Simoa HD-1 and HD-X Analyzer (Quanterix, Billerica, Mass.). These instruments employ a sandwich enzyme-linked immunosorbent assay (ELISA) format. In brief, sandwich complexes are formed by (1) capturing target molecules on antibody-coated paramagnetic beads; (2) binding the captured target with a biotinylated detection antibody, and (3) labeling the complex with streptavidin-β-galactosidase (SβG). The beads are then individually loaded onto a microarray of femtoliter wells with enzyme substrate, oil-sealed, and imaged. Average enzymes per bead (AEB) is the standard unit for Simoa assays. Details about the preparation of capture antibody-coated beads and biotinylated detection antibody, assay protocols and sample handlings are provided above.

### Clinical Samples and Study Design

All urine samples were provided by the FIND TB sample repository. FIND collected these samples under IRB/IEC approved studies in participating countries. Urine samples were collected from adult subjects with symptoms suggestive of pulmonary TB in South Africa, Peru, Vietnam, and Cambodia between June 2012 and February 2019, and all samples were collected prior to medical intervention. All clinical data were anonymized, and barcodes were used to access pertinent information. FIND uses standardized protocols for collecting and processing samples, which were reported previously in detail (Broger T, et al., PLoS One 2019; 14: e0215443; Sigal G B, et al., J Clin Microbiol 2018; 56: e01338-18). Obtained subject samples were categorized based on microbiological assessment. TB-positive individuals were patients with at least one positive culture result. Individuals categorized as NonTB were smear-negative and culture-negative from all sputum samples, negative for GeneXpert if tested, and their symptoms were improved or recovered in the absence of TB treatment at the follow-up symptom screening. The subjects were also classified as HIV-positive or HIV-negative on the basis of HIV rapid tests.

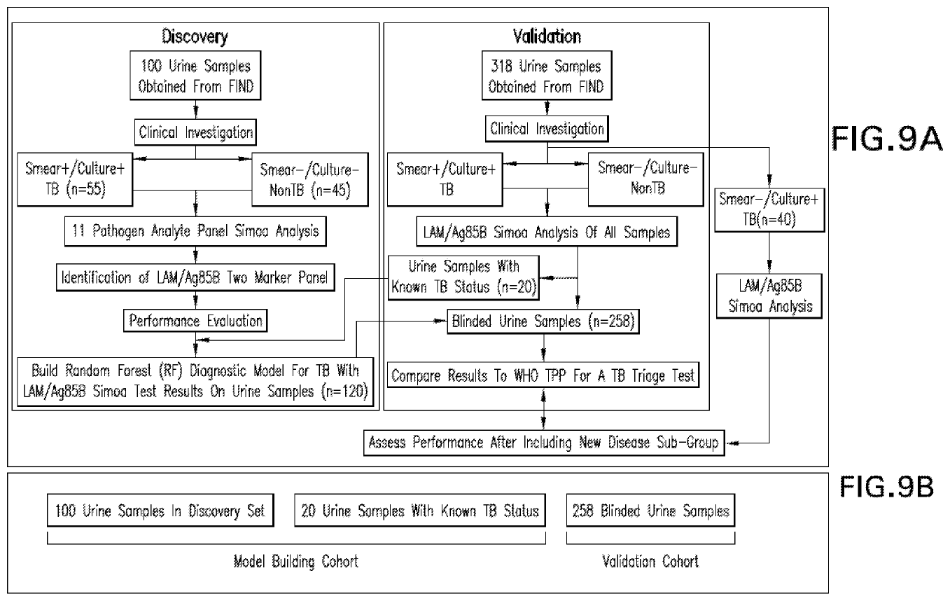

The study design flowchart is shown in FIG. 9A, and sample cohort allocation is shown in FIG. 9B. Demographic information of all patients is summarized in Table 5. This retrospective study was conducted in two major phases: 1) the discovery and model building phase, and 2) the blinded validation phase, and followed by an exploratory evaluation of model performance with addition of culture-confirmed smear-negative TB patient urine samples.

The discovery sample set included a total of 100 urine samples, which were collected from 45 NonTB individuals (25 HIV−, 20 HIV+), and 55 TB patients (20 HIV−, 35 HIV+). These samples were tested for all the 11 pathogen markers and were used to identify the most informative marker panel, consisted of LAM, Ag85B, and HIV-status. 20 additional samples were later measured for this panel and included (5 NonTB/HIV−, 5 NonTB/HIV+, 5 TB/HIV−, 5 TB/HIV+), and together this set of 120 samples, assigned as model building set, was used to build a diagnostic model based on the identified markers.

The validation phase was conducted using 258 urine samples subsequently obtained from FIND upon identification of the marker panel, of which the TB status was blinded until the classification results were reported to FIND. The validation set included 130 NonTB individuals (65 HIV+, 65 HIV−) and 128 TB patients (65 HIV+, 63 HIV−), randomly selected by FIND. These samples were tested for LAM and Ag85B and assessed by the diagnostic model. Classification performance was evaluated after TB status was unblinded by FIND.

For these two phases, the included TB patients all had positive sputum smear results. To explore model performance when smear-negative TB patients are included, LAM and Ag85B detection was then performed on an additional set of urine samples collected from 40 smear-negative culture-confirmed TB patients. Model performance was further assessed using the entire sample set combining these smear-negative samples with previous samples from the two major study phases.

TABLE 5
Clinical and demographic characteristics of all patients

| | | | | | | | | | | | | | Sputum Smear | |
|---|---|---|---|---|---|---|---|---|---|---|---|---|---|---|
| | Model Building Cohort | | | | | | Validation Cohort | | | | | | Negative TB | |
| | TB | | Non-TB | | All | | TB | | Non-TB | | All | | All | |
| | (n = 65) | | (n = 55) | | (n = 120) | | (n = 128) | | (n = 130) | | (n = 258) | | (n = 40) | |
| Age, years | 32 | (28-38) | 35 | (26-41) | 34 | (27-39) | 35 | (27-41) | 35 | (30-45) | 35 | (28-42) | 43 | (33-61) |
| Sex | | | | | | | | | | | | | | |
| Male | 46 | (71%) | 19 | (35%) | 65 | (54%) | 86 | (67%) | 52 | (40%) | 138 | (53%) | 25 | (62%) |
| Female | 19 | (29%) | 36 | (65%) | 55 | (46%) | 42 | (33%) | 78 | (60%) | 120 | (47%) | 15 | (38%) |
| Country | | | | | | | | | | | | | | |
| South | 24 | (37%) | 27 | (49%) | 51 | (43%) | 66 | (52%) | 76 | (58%) | 142 | (55%) | 23 | (58%) |
| Africa | | | | | | | | | | | | | | |
| Peru | 24 | (37%) | 17 | (31%) | 41 | (34%) | 46 | (36%) | 19 | (15%) | 65 | (25%) | 3 | (8%) |
| Vietnam | 16 | (25%) | 2 | (4%) | 18 | (15%) | 8 | (6%) | 22 | (17%) | 30 | (12%) | 5 | (12%) |
| Cambodia | 1 | (1%) | 9 | (16%) | 10 | (8%) | 8 | (6%) | 13 | (10%) | 21 | (8%) | 9 | (22%) |
| HIV status | | | | | | | | | | | | | | |
| Positive | 40 | (62%) | 25 | (45%) | 65 | (54%) | 65 | (51%) | 65 | (50%) | 130 | (50%) | 20 | (50%) |
| Negative | 25 | (38%) | 30 | (55%) | 55 | (46%) | 63 | (49%) | 65 | (50%) | 128 | (50%) | 20 | (50%) |
| HIV CD4 | 76 | (23-170) | 319 | (139-568) | 131 | (52-355) | 200 | (54-389) | 256 | (161-479) | 234 | (99-437) | 171 | (72-418) |
| Count | | | | | | | | | | | | | | |
| CD4 Count | 3 | (8%) | 3 | (12%) | 6 | (9%) | 13 | (20%) | 3 | (5%) | 16 | (6%) | 3 | (15%) |
| Unknown | | | | | | | | | | | | | | |

Data are in median (IQR), or n (%)

### Data Analysis

All undetectable samples, i.e. samples with concentration readings below the limit of detection (LOD), were assigned a value equal to the sample LOD of the corresponding assay to be included in the analysis. For analyses, a p-value of less than 0.05 was considered statistically significant. Mann-Whitney tests were used to compare the difference between groups. Correlation constants and p-values for the null hypothesis of no correlation were calculated using spearman rank correlation test. All data points, including those assigned with LOD values, were included in these analyses.

Before model development, data were log transformed and normalized to zero mean and unit variance. In the discovery phase, antigens with concentrations below the LOD in >50% of the TB group samples or those that had no significant difference between TB and NonTB groups were filtered out. The remaining antigens were used to construct the model. A Random Forest (RF) machine learning algorithm with nested 10-fold cross-validation was used to evaluate the preliminary performance in the discovery sample set, and to construct the final model for classification using the model building sample set. This model was then used to classify the blinded validation samples.

To explore the impact of including smear-negative TB patients (N=40) on the model performance, two approaches were applied. First, the RF algorithm was used with 10-fold cross-validation on the entire sample cohort (N=418) to evaluate the performance. A second approach split the smear-negative samples while maintaining the same ratio between the model-building and validation cohorts, and then added to each cohort respectively (N=14 added to model-building, and N=26 added to validation cohorts). Model training and testing was subsequently performed.

The accuracy of the classification was evaluated by area under receiver operating characteristic curves (ROC-AUC). Confidence intervals (CI) for ROC curves' sensitivities were plotted at 1% specificity intervals. ROC-AUCs were compared using pairwise approach with the DeLong tests. For blinded validation, sensitivity and specificity were derived from the classification results. The accuracy was assessed when fixing sensitivity and specificity at the minimum and optimal thresholds defined by WHO TPP TB triage test requirements, which were minimum 90% sensitivity, 70% specificity; optimum 95% sensitivity, 80% specificity 8. CIs for proportions were calculated using the binomial Wilson method.

To assess the statistical power, a published model was used for estimation of sample size in diagnostic tests (Hajian-Tilaki K. J Biomed Inform 2014; 48: 193-204). Case-control design was employwed in this study and balanced samples in TB and NonTB groups were used, thus the prevalence of TB in all subjects was 50%. At this prevalence, a total of 276 participants would achieve the minimum sensitivity threshold of the WHO TPP for a triage test (at 90%) with a 5% margin of error. The available samples included in the blinded validation was 258, which according to the estimation, yielded a precision at 5.2% margin of error.

Reporting of this study follows STARD guidelines (Bossuyt P M, et al. Radiology 2015; 277: 826-32.). RF model was developed in Python (version 3.7.6). All the other statistical analyses, and data visualization were done in R (version 3.6.3) or GraphPad Prism (version 8.4.2).

### Detection of Biomarker Candidates in Urine and the Initial Evaluation

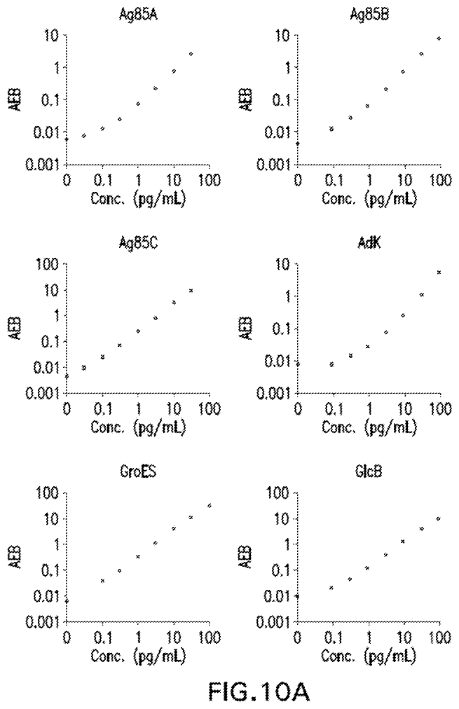

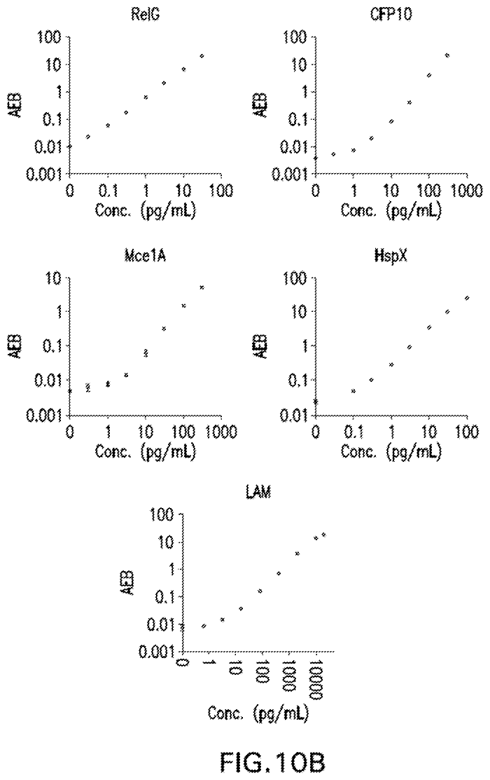

11 M. tb antigen biomarker candidates were selected. Digital ELISAs based on Simoa assays were developed for these candidates with pg/mL to sub-pg/mL analytical sensitivity and all assays were validated to ensure analytical robustness (FIGS. 10A-B and Table 6).

TABLE 6
TB biomarker Simoa assay characteristics.

| | LOD | Average Recovery % | Average Dilution |
|---|---|---|---|
| Marker | (pg/mL) | (min-max) | Linearity Fitted R² |
| Ag85A | 0.017 | 107 (92-130) | >0.99 |
| Ag85B | 0.015 | 109 (86-146) | 0.99 |
| Ag85C | 0.001 | 114 (95-138) | >0.99 |
| Adk | 0.16 | 111 (70-175) | 0.99 |
| GroES | 0.015 | 101 (75-141) | 0.98 |
| GlcB | 0.025 | 109 (82-135) | >0.99 |
| RelG | 0.004 | 119 (99-168) | >0.99 |
| LAM | 5.91 | 90 (70-113) | 0.98 |
| CFP10 | 0.56 | 86 (52-155) | 0.98 |
| Mce1A | 0.70 | 94 (36-162) | 0.94 |
| HspX | 0.069 | 88 (19-162) | >0.99 |

LOD: limit of detection

Using these Simoa assays, a discovery cohort (Table 7 and Table 8) of urine samples was first tested. Despite the high sensitivity offered by Simoa, a majority of these biomarkers had low detectability in TB patients (>50% not detectable). After filtering markers to remove those with over 50% undetectable values in TB samples, the remaining markers were LAM, Ag85B, and Ag85C. Furthermore, a Mann-Whitney test showed that concentration differences of Ag85C were not significant (p=0.094) to distinguish TB from NonTB, thus it was excluded from further analysis (Table 8). LAM and Ag85B3 concentrations were both significantly higher in TB patients (both with p<0.0001), and therefore they passed initial marker filtration to be used to construct the classification model.

TABLE 7
Clinical and demographic characteristics of patients in the
discovery cohort.

| | Discovery Cohort | | | | | |
|---|---|---|---|---|---|---|
| | TB (n = 55) | | Non-TB (n = 45) | | All (n = 100) | |
| Age, years | 31 | (28-38) | 35 | (27-40) | 34 | (27-39) |
| Sex | | | | | | |
| Male | 41 | (75%) | 16 | (36%) | 57 | (57%) |
| Female | 14 | (25%) | 29 | (64%) | 43 | (43%) |
| HIV status | | | | | | |
| Positive | 35 | (64%) | 20 | (44%) | 55 | (55%) |
| Negative | 20 | (36%) | 25 | (56%) | 45 | (45%) |
| HIV CD4 Count | 76 | (21-186) | 319 | (129-584) | 128 | (49-355) |

| | | | |
|---|---|---|---|
| CD4 Count | — | — | — |
| Unknown | | | |

Data are in median (IQR), or n (%)

TABLE 8
Detectability of 11 TB markers in the discovery cohort.

| | | Number of detectable | | | | | Include/ |
|---|---|---|---|---|---|---|---|
| | | samples | | | | | Exclude |
| | LOD | TB | | NonTB | | | for model |
| Antigen | (pg/mL) | (n = 55) | | (n = 45) | | p value | construction |
| LAM | 5.91 | 53 | (96%) | 13 | (29%) | <0.0001 | Include |
| Ag85B | 0.015 | 45 | (82%) | 19 | (42%) | <0.0001 | Include |
| Ag85C | 0.001 | 37 | (67%) | 22 | (49%) | 0.094 | Exclude |
| Ag85A | 0.017 | 13 | (24%) | 22 | (49%) | — | Exclude |
| Adk | 0.16 | 9 | (16%) | 13 | (29%) | — | Exclude |
| GroES | 0.015 | 6 | (11%) | 4 | (9%) | — | Exclude |
| GlcB | 0.025 | 2 | (4%) | 6 | (13%) | — | Exclude |
| RelG | 0.004 | 5 | (9%) | 8 | (18%) | — | Exclude |
| CFP10 | 0.56 | 15 | (27%) | 7 | (16%) | — | Exclude |
| Mce1A | 0.70 | 6 | (11%) | 9 | (20%) | — | Exclude |
| HspX | 0.069 | 1 | (2%) | 1 | (2%) | — | Exclude |

Data are n (%). Mann-Whitney test was used to test significance of difference between TB and NonTB patients with Bonferroni corrections. Undetectable samples were assigned a value at assay limit of detection (LOD) to be included in the analysis.

A Random Forest (RF) machine learning algorithm was used with nested 10-fold cross-validation to assess the abilities of biomarker panels composed of different combinations of LAM, Ag85B3, and HIV-status to discriminate between TB and NonTB individuals (Table 9). HIV-status was included because previous evidence has shown that TB patients with HIV coinfection tended to shed higher levels of LAM into urine (Paris L, et al., Sci Transl Med 2017; 9: eaa12807; Sigal G B, et al., J Clin Microbiol 2018; 56: e01338-18; MacLean E, et al., Clin Chem 2018; 64: 1133-5). It was hypothesized that HIV-status could play a role in affecting the biomarker concentrations in TB patient urine samples and thus affect the classification performance. In this initial analysis, LAM alone provided an average ROC-AUC of 0.907 [standard error (S.E.) 0.003] using the 10-fold cross-validation, while Ag85B3 alone gave an average ROC-AUC of 0.861 (S.E. 0.003). Improved accuracy was achieved by combining LAM and Ag85B3 as a two-marker panel, with the average ROC-AUC of 0.937 (S.E. 0.005). When adding HIV-status to this panel, a slight improvement in accuracy was observed, showing an average ROC-AUC of 0.941 (S.E. 0.025), which preliminarily confirmed the hypothesis of HIV-status effects. This three-marker panel, consisting of LAM, Ag85B, and HIV-status was subsequently used to develop the classification model to distinguish TB from NonTB samples.

TABLE 9
Performance metrics of LAM and Ag85B based signatures in the
discovery cohort*.

| | | | | NonTB | ROC-AUC |
|---|---|---|---|---|---|
| Marker Panel | Cohort | All (n) | TB (n) | (n) | (S.E.) |
| LAM | Discovery* | 100 | 55 | 45 | 0.916 (0.004) |
| Ag85B | ″ | ″ | ″ | ″ | 0.872 (0.004) |
| LAM, Ag85B | ″ | ″ | ″ | ″ | 0.937 (0.005) |
| LAM, Ag85B, | ″ | ″ | ″ | ″ | 0.941 (0.025) |
| HIV | | | | | |

*Demographic information of discovery cohort in Table 7. Performance of different marker combinations based on Random Forest (RF) model with nested 10-fold cross-validation.
S.E.: standard error.

### Development of Urine Three-Marker Model for Detection of Active TB

To develop a classification model for TB based on identified biomarkers as well as to assess and validate its performance, a second batch of urine samples was obtained from FIND, which included an additional 20 urine samples (10 TB and 10 NonTB), as well as a blinded sample set (n=258), which were preserved for model validation (FIGS. 9A and 9B, validation see next subsection). The 20 samples were measured for LAM and Ag85B using the Simoa assays, and concentration differences were observed between TB and NonTB groups for both markers (Mann-Whitney test p=0.0014 for LAM, and p=0.0049 for Ag85B). This observation supported the initial identification of LAM and Ag85B as informative markers for TB detection. Model development was then proceeded, using a sample cohort consisting of the discovery sample set plus these 20 samples, designated as the model building cohort (n=120, FIG. 9B).

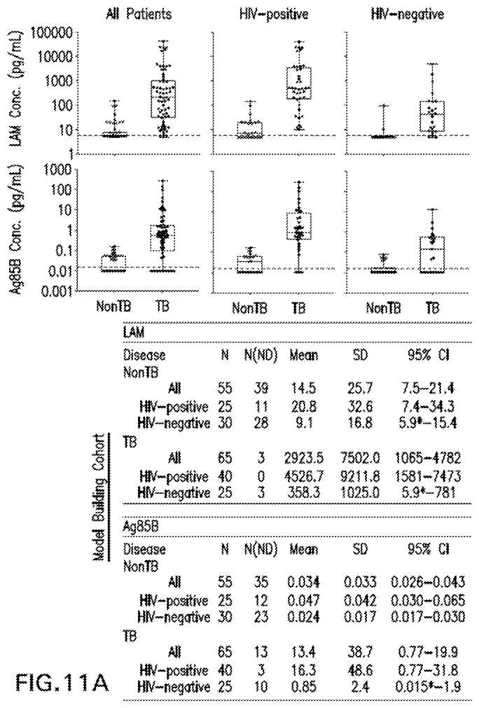

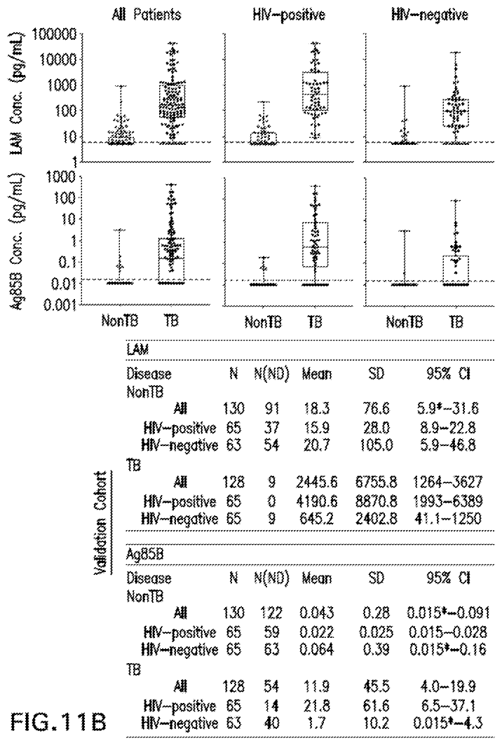

As shown in FIG. 11A, the corresponding box plots show distinct separation between TB and NonTB patients in the entire model building cohort and also in the HIV-stratified subsets. The majority of the NonTB samples had undetectable levels of biomarkers (71% for LAM, and 64% for Ag85B). This result was expected for TB pathogen markers that are highly specific to infection. Only 5% and 20% of the TB samples had undetectable levels of LAM and Ag85B, respectively. Most of the undetectable samples in the TB group were patients without HIV coinfection. Notably, all HIV-positive TB samples had detectable levels of LAM. In order to include all patients in the analysis, undetectable samples were assigned a value equal to the LOD of the corresponding assay. The concentration differences between TB and NonTB samples were significant for both LAM and Ag85B (Mann-Whitney test p=0.007 for Ag85B in HIV-negative subset, all others p<0.0001). Overall, the mean concentrations of LAM were over 200-fold higher in TB samples than in NonTB samples, and Ag85B showed a greater than 300-fold difference. Differences were less distinct in the HIV-negative subset, but still maintained over 30-fold higher concentrations in TB than in NonTB samples for both markers.

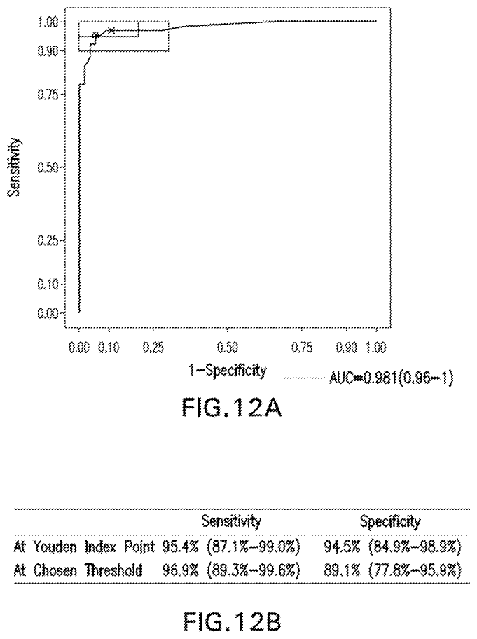

As described above, a three-marker RF classification model based on LAM, Ag85B, and HIV-status was then developed using the model building cohort. It yielded a training ROC-AUC of 0.981 (95% CI, 0.96-1), which represented an upper limit of its performance. When benchmarked against the WHO TPP criteria for a TB triage test, the ROC curve partially lies in the region defined by the optimal cutoffs of 95% sensitivity and 80% specificity (FIG. 12A). Since the objective is to develop a TB triage test, the classification should ideally maximize the sensitivity while maintaining an acceptable specificity. In line with this objective, instead of using a cutoff value at the maximum Youden index which reflects the overall highest accuracy (Youden W J. Cancer 1950; 3: 32-5), an optimal threshold was determined, to a) correctly classify all TB patients coinfected with HIV in the model building cohort without any false rule-out, and b) further boost sensitivity while maintaining a specificity that is above WHO TPP optimal criteria for a triage test. This threshold yielded a model sensitivity of 96.9% (95% CI, 89.3-99.6%), and a specificity of 89.1% (95% CI, 77.8-95.9%) (FIG. 12B). At this sensitivity (96.9%), the specificity decreased drastically from 90% to 72% (FIG. 12A). The optimal threshold that could meet both requirements with the highest specificity was then selected.

### Validation of Model Performance in Blinded Samples

To validate the model and further interrogate its performance, a blinded set of samples was obtained from FIND and used as the validation cohort. Simoa LAM and Ag85B analyses were applied to the validation cohort. After submitting the assay and classification results to FIND, the unblinded sample codes were received. The mean and SD of biomarker concentration measurements and their corresponding box plots are shown in FIG. 11B. Clear separation between TB and NonTB patients were again observed overall and in the HIV stratified subsets. The concentration differences between TB and NonTB samples was significant for both LAM and Ag85B (Mann-Whitney test p<0.0001). Similarly, LAM was undetectable in 70% of the NonTB samples but only 7% of the TB samples, and the Simoa LAM assay again detected all TB patients with HIV coinfection. Compared to the model building cohort, a higher proportion of undetectable samples was found for Ag85B, with 94% in NonTB and 42% in TB. Nevertheless, the mean concentrations of LAM maintained a greater than 100-fold difference between NonTB and TB patients, while Ag85B maintained a greater than 200-fold difference.

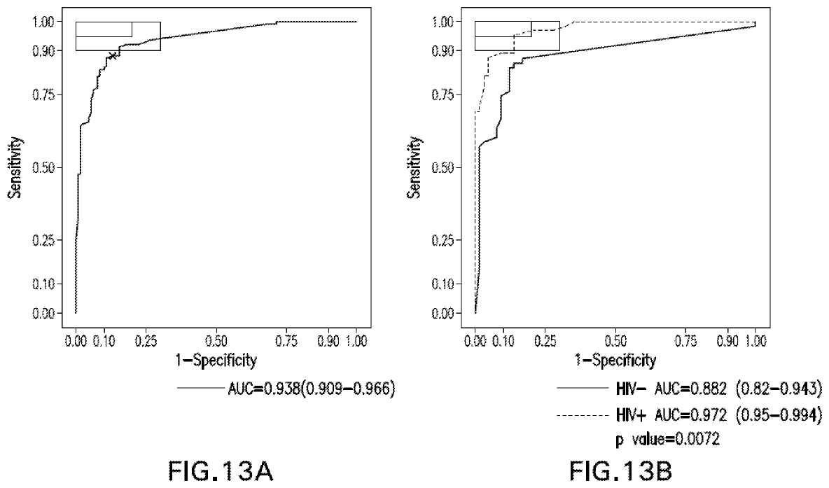

The three-marker RF model applied to the blinded validation cohort provided a classification ROC-AUC of 0.938 (95% CI, 0.909-0.966), with 88.3% sensitivity (95% CI, 81.4-93.3%) and 86.9% specificity (95% CI, 79.9-92.2%) (Table 10). The ROC curve is shown in FIG. 13A. During a subgroup analysis, the model showed performance was affected by HIV infection (DeLong ROC comparison p=0.0072) (FIG. 13B), with a better performance in the HIV-positive subgroup (ROC-AUC of 0.972, 95% CI, 0.95-0.994) versus the HIV-negative subgroup (ROC-AUC of 0.882, 95% CI, 0.82-0.943). The major impact observed was on specificity, showing a 7.7% difference between HIV-positive and HIV-negative subgroups (Table 10).

TABLE 10
Model performance characteristics in the blinded
validation cohorts.

| | Sensitivity | Specificity | ROC-AUC |
|---|---|---|---|
| All (N = 258) | | | |
| % (n/N) | 88.3 | 86.9 | 0.938 |
| | (113/128) | (113/130) | |
| 95% CI | (81.4-93.3) | (79.9-92.2) | (0.909-0.966) |
| HIV-positive (N = 130) | | | |
| % (n/N) | 89.2 | 90.8 | 0.972 |
| | (58/65) | (59/65) | |
| 95% CI | (79.1-95.6) | (81.0-96.5) | (0.950-0.994) |
| HIV-negative (N = 128) | | | |
| % (n/N) | 87.3 | 83.1 | 0.882 |
| | (55/63) | (54/65) | |
| 95% CI | (76.5-94.4) | (71.7-91.2) | (0.820-0.943) |

The performance was further evaluated using WHO TPP criteria for a TB triage test as a reference (WHO. High-priority target product profiles for new tuberculosis diagnostics: report of a consensus meeting. Geneva, Switzerland, 2014). First, at the current test cutoff, the results approximated the minimum sensitivity criteria of 90% with an optimal specificity at 86.9% (Table 11). Then, when the ROC curve was benchmarked against TPP criteria, point estimates reached the minimum threshold at 90% sensitivity and 70% specificity, while the 95% CI reached the optimal cutoff at 95% sensitivity and 80% specificity (FIG. 13A). Furthermore, when the thresholds were fixed to enforce either sensitivity at 90% or specificity at 70%, the model met the required minimum performance thresholds. For the optimal requirements, when setting the threshold to enforce specificity at 80%, the model was approximated to the required 95% sensitivity. However, it failed to meet the required 80% specificity when fixing sensitivity at 95%. With a 63.6% (95% CI, 54.7-71.8%) specificity, the model fell short of the optimal performance requirement.

The sample set was mostly balanced for TB disease, and the NonTB controls were patients with TB-like symptoms rather than healthy individuals. It was found to be reasonable to assume a 5% TB prevalence among people seeking care to calculate the positive and negative predictive values (PPV and NPV), which would be more reflective under the healthcare settings in high TB burden countries. The calculated PPV and NPV were 26.2% (95% CI, 22.1-30.6%) and 99.3% (95% CI, 98.8-99.6%) respectively (Table 11).

TABLE 11
Model performance metrics in the blinded validation cohort
benchmarked against WHO TPP requirements.

| Validation set | | | PPV (at 5% | NPV (at 5% |
|---|---|---|---|---|
| (N = 258) | Sensitivity | Specificity | prevalence) | prevalence) |
| Current test | 88.3% | 86.9% | 26.2% | 99.3% |
| | (81.4-93.3) | (79.9-92.2) | (22.1-30.6) | (98.8-99.6) |
| At minimum | 90% | 84.6% | 23.5% | 99.4% |
| sensitivity for a triage | | (77.2-90.3) | (19.8-27.6) | (98.9-99.7) |
| test | | | | |
| At minimum | 94.1% | 70% | 14.2% | 99.6% |
| specificity for a triage | (88.6-97.5) | | (11.9-16.7) | (99.1-99.8) |
| test | | | | |
| At optimal sensitivity | 95% | 63.6% | 12.1% | 99.6% |
| for a triage test | | (54.7-71.8) | (10.1-14.2) | (99.1-99.8) |
| At optimal specificity | 92.2% | 80% | 19.5% | 99.5% |
| for a triage test | (86.1-96.2) | | (16.4-22.9) | (99.1-99.8) |

Data are % (95% CI). WHO target product profile (TPP) requires a TB triage test with minimum 90% sensitivity and 70% specificity, and optimal 95% sensitivity and 80% specificity.

### Impact of Marker and Sample Variance on Model Performance

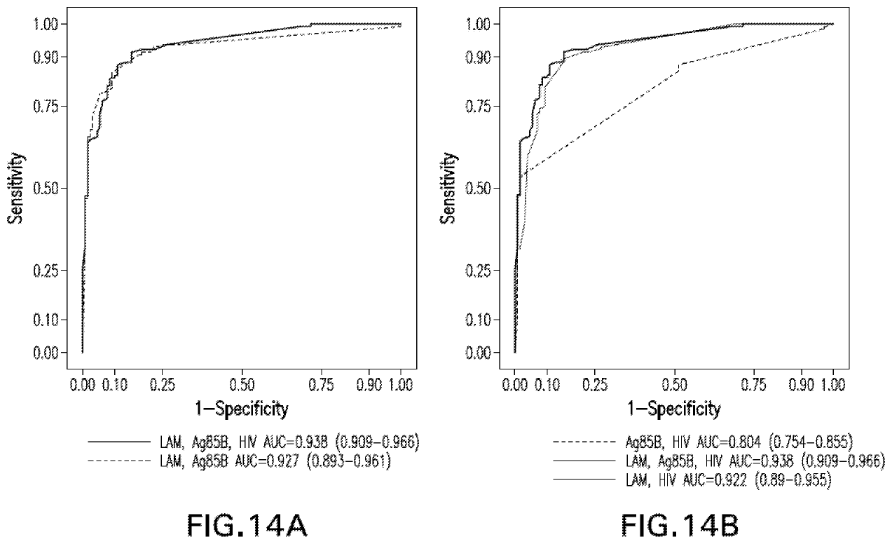

The performance by reducing the number of markers in the model was also explored. An approach to retrain models with designated markers was taken using the model building cohort, and the performance was tested in the validation cohort. A two-marker model consisting of LAM and Ag85B yielded a ROC-AUC of 0.927 (95% CI, 0.893-0.961), slightly lower (−0.011) than the original model where HIV-status was included (FIG. 14A), which was consistent with results from the discovery cohort (Table 9). Omitting Ag85B from the panel provided a 0.922 ROC-AUC (95% CI, 0.890-0.955), showing a slight decrease (−0.016) from the original model (FIG. 14B). However, when LAM was omitted, an obvious decrease in accuracy was observed (−0.134 in ROC-AUC), suggesting that LAM plays a major role in determining the model classification performance. These results suggest that while the full three-marker model provided optimal performance, a simplified assay with fewer markers could reduce costs while maintaining strong performance.

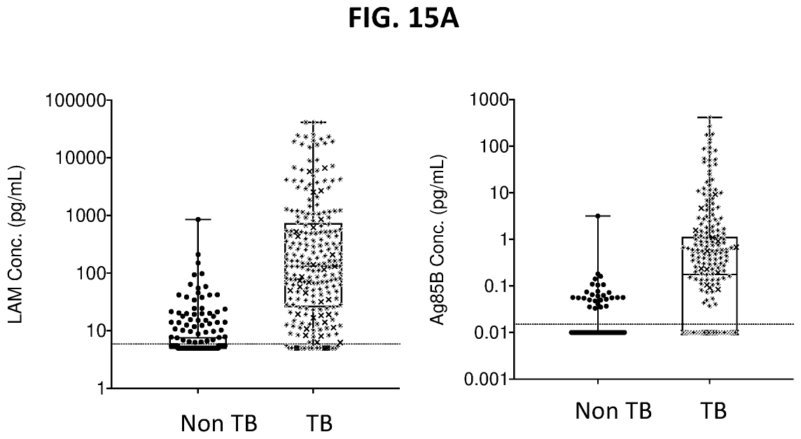

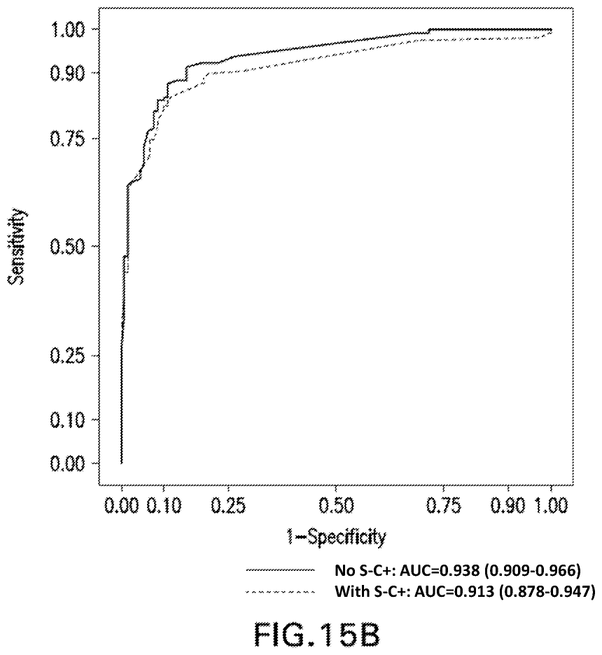

Thus far, the panel identification and model development were established to distinguish between culture-confirmed TB patients with positive sputum-smear and NonTB individuals. To gain a better knowledge of the utility of this model in a wider spectrum of TB conditions, the impact on model performance was further evaluated by including smear-negative TB patients both with and without HIV coinfection. FIG. 15A shows the corresponding boxplot of all patients in the study including the smear-negative TB subgroup; the mean concentrations of LAM and Ag85B in the smear-negative TB subgroup are shown in Table 12. For both markers, mean concentrations in the smear-negative subgroup are higher than those in NonTB individuals (22.8-fold for LAM, and 12.4-fold for Ag85B), and lower than those in smear-positive TB patients (4.9-fold for LAM, and 31-fold for Ag85B). Two approaches were applied to explore the impact on the model performance. First, as an initial evaluation, a RF machine learning algorithm with nested 10-fold cross-validation was applied to the entire patient cohort (N=418) and provided a cross-validation ROC-AUC of 0.907 (S.E. 0.017). The second approach split the smear-negative samples while maintaining the same ratio between the original model building and validation cohorts, and then separately added to the model building cohort (N=14 smear-negative added to reach a total N=134) to retrain the three-marker model, and to the validation cohort (N=26 smear-negative added to reach a total N=284) to test the classification performance of the retrained model. The resulting ROC-AUC was 0.913 (95% CI, 0.878-0.947) (FIG. 15B), which was close to the performance from cross-validation in the first approach but showed a slight decrease (−0.025) as compared to the performance in blinded validation without smear-negative TB patients. Similarly, when benchmarked against WHO TPP criteria, point estimates of the ROC curve met the minimum requirement for a TB triage test. This was also confirmed by enforcing the sensitivity at 90% and observing a resulting specificity at 78.1% (95% CI, 70.1-84.9%), or enforcing the specificity at 70% and observing a resulting sensitivity at 90.8% (95% CI, 85.1-94.8%); both models reached the minimum thresholds defined by TPP.

TABLE 12
Concentrations of LAM and Ag85B in the smear-negative TB patient
urine samples

| | N | N(ND) | Mean | SD | 95% CI |
|---|---|---|---|---|---|
| LAM | | | | | |
| All S-C+ | 40 | 9 | 532.9 | 1446.7 | 70.2-995.6 |
| HIV-positive | 20 | 4 | 948.1 | 1969.2 | 26.5-1869.8 |
| HIV-negative | 20 | 5 | 117.6 | 235.3 | 7.51-227.7 |
| Ag85B | | | | | |
| All S-C+ | 40 | 27 | 0.50 | 1.61 | 0.015*-1.01 |
| HIV-positive | 20 | 11 | 0.90 | 2.22 | 0.015*-1.94 |
| HIV-negative | 20 | 16 | 0.10 | 0.24 | 0.015*-0.21 |

S-C+: smear-negative TB.
N(ND): number of not detectable samples in each disease categories.
The mean concentrations were calculated after assigning a value at LOD to ND samples.
*95% CI lower limit below LOD values were replaced with values at LOD.

### Discussion

This study first assessed whether a urine-based pathogen signature for active TB could distinguish it from NonTB disease in patients with TB-like symptoms regardless of their HIV status, thereby providing a foundation for a TB triage test. Importantly, the sample cohort selected reflected the target population of a triage test, i.e. adults with symptoms suggestive of active pulmonary TB. Those subjects ruled out by a microbiological reference standard served as the NonTB controls, instead of healthy subjects who are not within the target population for the intended use of the test. Furthermore, these samples were collected from various countries with high burdens of TB and resources-constrained areas where a triage test is most needed.

In contrast to host markers that can be implicated in many infections and diseases, M. tb pathogen marker detection has the potential to be highly specific for TB. Evidence from previous studies has confirmed the presence of M. tb antigens in urine (Broger T, et al. Lancet Infect Dis 2019; 19: 852-61; MacLean E, et al. Clin Chem 2018; 64: 1133-5; Lawn S D, et al. Lancet Infect Dis 2012; 12: 201-9). However, detection of M. tb antigen markers remains challenging due to their low abundance in body fluids paired with insufficient sensitivity of current analytical techniques. Only a limited number of studies have investigated levels of pathogen markers in non-sputum body fluids and their potential for use in diagnostic tests. However, their classification performances, if reported, were not validated. Using ultrasensitive digital immunoassays with limits of detection in the pg/mL to sub-pg/mL range, the inventors were able to access and then quantify M. tb antigens in human urine. The study started with a panel of 11 pathogen markers in the discovery phase. LAM, Ag85B, and Ag85C presented detectable levels in over 50% of TB patients. Eight of the 11 markers (except McelA, CFP10, and LAM) showed urinary levels below 1 pg/mL for over 90% of the detectable samples. These antigens were measurable due to the high analytical sensitivity provided by Simoa technology. However, many antigens remained undetectable, suggesting that they may be either absent from urine (or present at very low levels, below our assay LODs), or that antigens were present in a form that was inaccessible using the sandwich ELISA format.

The final panel consisted of three markers of LAM, Ag85B, and HIV-status. Ag85B is strongly immunogenic in mycobacterial infections and is a major secreted marker of TB. As part of the Ag85 complex family, many attempts have been made to explore its diagnostic utility; however, detection in blood or urine showed highly variable performances and its diagnostic potential as a standalone marker remains unclear (Phan L M T, et al. Sensors Actuators, B Chem 2020; 317: 128220; Garcia-Basteiro A L, et al. Rev Port Pneumol 2018; 24: 73-85). In contrast, LAM has been implemented into the AlereLAM, a POC format lateral flow assay, which has been endorsed by WHO for the limited indication of HIV-positive patients with low CD4 counts. This limited usage is largely due to insufficient analytical sensitivity, and the AlereLAM assay is intended to be used only as a complement to GeneXpert. The recently developed next-generation FujiLAM POC test with a LOD of around 30 pg/mL, which is approximately 30-fold lower than the AlereLAM, showed superior performance with overall sensitivity of 71% and specificity of 96% in diagnosing TB patients with HIV coinfection (Broger T, et al. Lancet Infect Dis 2019; 19: 852-61). While the utility of FujiLAM in the HIV-negative population is undergoing investigation, a few recent studies explored LAM detection in HIV-negative patients. These studies confirmed the presence of LAM in the urine of TB patients with no HIV coinfection and showed potential for discriminating between TB and NonTB; however, they either required a pre-concentration step that may hinder its path toward POC adoption, or needed additional performance validation. Their findings suggested that a lower LOD can yield higher diagnostic sensitivity and again confirmed the need to improve the assay's analytical sensitivity (Paris L, et al. Sci Transl Med 2017; 9: eaa12807; Sigal G B, et al. J Clin Microbiol 2018; 56: e01338-18).

Consistent with these previous findings, the results from blinded validation showed that LAM concentrations tended to be lower in HIV-negative TB patients; similar results were also observed for Ag85B. Concentration differences depending on HIV status likely led to the significant difference of classification performance in patients with or without HIV coinfection. In this study, a value equal to the Simoa assay LOD was assigned to those samples with an undetectable biomarker level. While this approach ensured inclusion of all individuals for model development and evaluation, missing the lower end of the full dynamic range of analytes in patients could partly contribute to the overall performance reduction as well as the decrease in specificity in the HIV-negative subgroup compared to the HIV-positive subgroup. As a higher number of TB patients in the HIV-negative subgroup had undetectable levels of biomarkers and were assigned a value at the LOD, concentration differences between TB and NonTB were thus less distinct. While Simoa offers assay LODs down to 5.9 pg/mL and 0.015 pg/mL for LAM and Ag85B, respectively, further improving assay analytical sensitivity should improve the overall classification performance as some of the samples which were undetectable will become measurable thus providing further insights into the full dynamic range of these biomarkers in all patients. Overall, the blinded validation showed a performance that approximated the WHO TPP minimum TB triage criteria, regardless of HIV status. This performance was retained when including smear-negative TB patients in the evaluation, with point estimates identical to the WHO TPP threshold value.

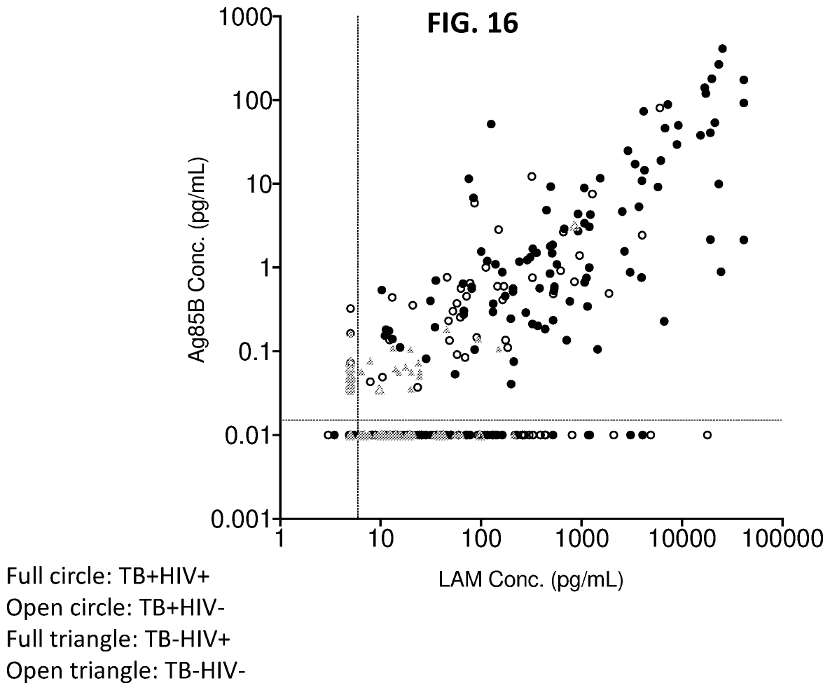

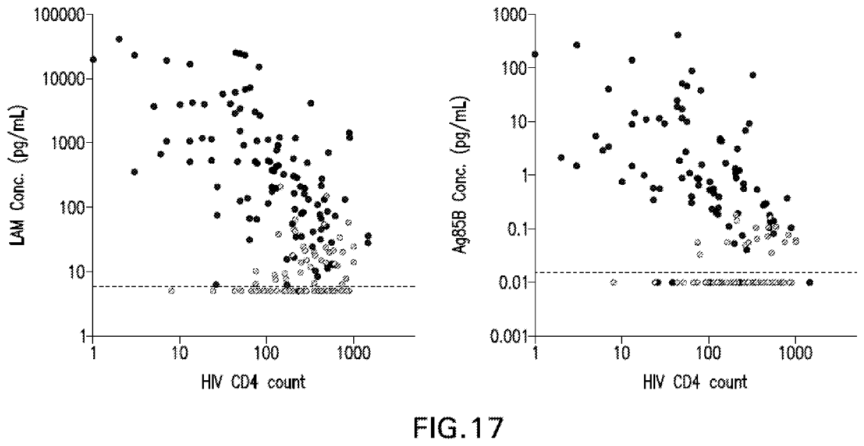

As part of the findings, concentrations of LAM and Ag85B in urine were positively correlated (Spearman coefficient 0.68, p<0.001, FIG. 16), and negative correlations between CD4 cell count and LAM or Ag85B were observed (FIG. 17). These findings demonstrated that urine LAM concentration increases with higher mycobacterial burden, and suggested that severely immunosuppressed patients tended to have high concentrations of both LAM and Ag85B in urine.

Besides the accuracy requirements, the key characteristics of a TB triage test include ease of use, affordability, and minimal infrastructure and training requirements, as defined by WHO TPP. A point of care (POC) format test could meet these needs in resource constrained areas. One approach that was taken toward the POC amendment was to narrow down the biomarker panel, which could reduce the test cost and technical complexity. In the post-hoc analysis, LAM remains the most promising target and played a major role in the model. Omitting HIV-status or Ag85B from the marker panel had a slight impact on the model performance. This result provides flexibility for further development of a simple assay to meet both practicality and cost requirements. In parallel, ongoing technology evolution would also facilitate this process. The current Simoa HD-X platform was designed to perform fully automated sample-to-result tests with high throughput and reproducibility in a central laboratory environment. With a per-test cost of approximately $10, this is beyond the POC target price range, however, Simoa employs a digital ELISA format, which is amenable to a POC format with less costly integrated consumables. This could allow the development of assays that meet the WHO targeted $2 per-test cost while still maintaining high analytical sensitivities by adopting its core concept of single molecule counting. Examples include platforms incorporating a smartphone-based imaging technique with droplet microfluidics or microbubbles. For example, the newly developed dropcast Simoa, a digital ELISA platform that only requires a microscope slide for bead loading to eliminate the need for complexed microwell or droplet fabrication, and a simple optical setup for signal readout, could be employed. Increased sampling efficiency during this process also enables the counting of more target molecules, which improves the detection limit to the attomolar range. Such design greatly simplified readout process and improved cost-effectiveness as well as assay sensitivity, which offers one promising path towards a POC diagnostic.

In conclusion, a TB test was developed using Simoa assays with high analytical sensitivity to quantify M. tb antigens in urine. A biomarker panel consisting of LAM, Ag85B, as well as HIV-status was subsequently identified and validated with a blind cohort to classify active TB patients in adults. The assay showed better performance in HIV-positive patients, and met minimum WHO TPP classification performance requirements for a TB triage test regardless of HIV status, suggesting the discovery of a broadly applicable panel which is not limited to HIV-positive individuals. The Simoa format also offers a clear path toward a cost-effective POC. Results from this study show promise for developing a triage test based on pathogen biomarkers in urine to detect active TB.

### EQUIVALENTS

Those skilled in the art will recognize, or be able to ascertain using no more than routine experimentation, many equivalents to the specific embodiments of the invention described herein. such equivalents are intended to be encompassed by the following claims. The contents of all references, patents and published patent applications cited throughout this application are incorporated herein by reference.

We claim:

1. A method for diagnosing a Mycobacterium tuberculosis infection in a subject, the method comprising
(a) contacting a subject sample suspected of containing an antigen expressed by Mycobacterium tuberculosis with a plurality of capture objects, wherein each capture object comprises a capture probe coupled to a plurality of capture moieties that specifically bind to the antigen, wherein the subject sample and the plurality of capture objects are contacted at a ratio that allows for a single antigen to bind to a single capture object;
(b) incubating the product of step (a) for a sufficient time to allow binding of the plurality of capture objects to the antigen contained in the sample, thereby creating a plurality of complexes of the capture object and the antigen;
(c) contacting the plurality of complex of the capture object and the antigen from step (b) with a plurality of detection probes, wherein the detection probes bind to the antigen within the complex of the capture object and the antigen from step (b);
(d) labeling the product of step (c) with a detectable moiety;
(e) detecting the detectable moiety;
(f) quantifying the concentration of the antigen in the subject sample by determining the number of detected moieties in step (e); and
(g) comparing the concentration determined in step (f) with a control value determined using a sample from a healthy subject, wherein an antigen concentration greater than the control value indicates the presence of Mycobacterium tuberculosis infection in the subject and an antigen concentration less than the control value indicates the absence of Mycobacterium tuberculosis infection in the subject,
thereby diagnosing the Mycobacterium tuberculosis infection in the subject.

2. The method of claim 1, wherein the Mycobacterium tuberculosis infection is an active infection, or a latent infection.

3. The method of claim 1, wherein the antigen molecule expressed by Mycobacterium tuberculosis is one or more antigens selected from the group consisting of lipoarabinomannan (LAM) and Ag85B (Rv1886c).

4. The method of claim 1, wherein each group of capture objects comprises a plurality of capture moieties that bind to a distinct epitope on the antigen and/or is distinguishably labeled by a fluorescent label or an enzymatic label.

5. The method of claim 1, wherein the capture moiety on the capture probe is a full-length antibody, an antigen-binding fragment of an antibody, or an antibody mimetic.

6. The method of claim 1, wherein the capture probe is selected from the group consisting of beads, nanotubes, and polymers.

7. The method of claim 6, wherein the capture probe is a paramagnetic bead.

8. The method of claim 1, wherein the detection probe is an antibody, an aptamer, an antibody mimetic, a polypeptide, a nucleic acid, a molecularly-imprinted polymer, a receptor, or a small molecule.

9. The method of claim 1, wherein the detectable moiety is, or comprises, an enzymatic label, a fluorescent label, a radioactive label, or a metal label.

10. The method of claim 9, wherein the enzymatic label is selected from the group consisting of beta-galactosidase (β-galactosidase), horseradish peroxidase, glucose oxidase, and alkaline phosphatase.

11. The method of claim 1, wherein step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

12. The method of claim 11, wherein the non-covalent affinity binding pair is biotin-streptavidin.

13. The method of claim 1, wherein the sample is selected from the group consisting of lymph, serum, plasma, whole blood, urine, saliva, semen, synovial fluid, cerebrospinal fluid, sputum, mucous, feces, and vaginal fluid.

14. The method of claim 13, wherein the sample is a urine sample.

15. The method of claim 1, wherein prior to contacting with the capture objects, the sample is subject to centrifugation only; heat treatment prior to centrifugation; or centrifugation prior to heat treatment.

16. The method of claim 1, further comprising determining the HIV status of the subject.

17. The method of claim 1, wherein the subject is a human subject.

18. The method of claim 1, wherein the subject is HIV negative (HIV−) or HIV positive (HIV+).

19. The method of claim 1, wherein the subject has a reduced level of CD4 cell count.

20. The method of claim 1, wherein the detection of step (e) comprises single-molecule detection of the detectable moiety.

21. The method of claim 20, wherein the detection of step (e) occurs in an array of microwells, wherein the microwells are capable of holding zero or one capture objects.

22. The method of claim 21, wherein the array is a QUANTERIX™ single molecule array (Simoa).
